SEQUENCE LISTING (19 sequences)

SEQ ID NO: 1 - DNA, 21 bp, synthetic construct
  source 1..21: mol_type = other DNA
actgagggagccttgaataca

SEQ ID NO: 2 - DNA, 20 bp, synthetic construct
  source 1..20: mol_type = other DNA
tgcagcattgttagcaggat

SEQ ID NO: 3 - DNA, 21 bp, synthetic construct
  source 1..21: mol_type = other DNA
  misc_feature 1..11: note = DNA
  misc_feature 12: note = RNA
  misc_feature 13..21: note = DNA
  modified_base 20: mod_base = OTHER; note = LNA, locked Cytosine nucleotide
actgagggagccttgaataca

SEQ ID NO: 4 - DNA, 24 bp, synthetic construct
  source 1..24: mol_type = other DNA
  modified_base 21: mod_base = OTHER; note = ribose Uracil nucleotide
caagatcacagattttgggctggc

SEQ ID NO: 5 - DNA, 18 bp, synthetic construct
  source 1..18: mol_type = other DNA
tttctcttccgcacccag

SEQ ID NO: 6 - DNA, 20 bp, synthetic construct
  source 1..20: mol_type = other DNA
  modified_base 11: mod_base = OTHER; note = ribose Uracil nucleotide
  modified_base 19: mod_base = OTHER; note = LNA, locked Adenine nucleotide
tgcagcattgttagcaggat

SEQ ID NO: 7 - DNA, 40 bp, synthetic construct
  misc_feature 1..9: note = DNA
  misc_feature 10: note = RNA
  misc_feature 11..40: note = DNA
  source 1..40: mol_type = other DNA
  modified_base 39: mod_base = OTHER; note = LNA, locked Cytosine nucleotide
agacggcatcatatgggttgcacgggtgccaatgtgatct

SEQ ID NO: 8 - DNA, 19 bp, synthetic construct
  source 1..19: mol_type = other DNA
tggctactaccgaagagct

SEQ ID NO: 9 - DNA, 41 bp, synthetic construct
  source 1..41: mol_type = other DNA
tctggcccagttcctaggtagtccagacgaattcgtggtgg

SEQ ID NO: 10 - DNA, 25 bp, synthetic construct
  source 1..25: mol_type = other DNA
ggactgagatctttcattttaccgt

SEQ ID NO: 11 - DNA, 40 bp, synthetic construct; identical to SEQ ID NO: 7
  source 1..40: mol_type = other DNA

SEQ ID NO: 12 - DNA, 45 bp, synthetic construct
  misc_feature 1..21: note = DNA
  misc_feature 22: note = RNA
  misc_feature 23..45: note = DNA
  source 1..45: mol_type = other DNA
accaatagcagtccagatgaccaaattggctactaccgaagagct

SEQ ID NO: 13 - DNA, 46 bp, synthetic construct
  misc_feature 1..22: note = DNA
  misc_feature 23: note = RNA
  misc_feature 24..46: note = DNA
  source 1..46: mol_type = other DNA
gttccttgaggaagttgtagcacgattgcagcattgttagcaggat

SEQ ID NO: 14 - DNA, 27 bp, synthetic construct
  misc_feature 1..11: note = DNA
  misc_feature 12..13: note = RNA
  misc_feature 14..27: note = DNA
  source 1..27: mol_type = other DNA
  modified_base 1: mod_base = OTHER; note = a modified by 6-FAM reporter dye
  modified_base 27: mod_base = OTHER; note = a modified by BHQ
agctggacttccctatggtgctaacaa

SEQ ID NO: 15 - DNA, 24 bp, synthetic construct
  source 1..24: mol_type = other DNA
gctttcgtggtattcttgctagtt

SEQ ID NO: 16 - DNA, 25 bp, synthetic construct
  source 1..25: mol_type = other DNA
gttaacaatattgcagcagtacgca

SEQ ID NO: 17 - DNA, 56 bp, synthetic construct
  misc_feature 1..32: note = DNA
  misc_feature 33: note = RNA
  misc_feature 34..56: note = DNA
  source 1..56: mol_type = other DNA
cgttaatagttaatagcgtacttctttttcttgctttcgtggtattcttgctagtt

SEQ ID NO: 18 - DNA, 53 bp, synthetic construct
  misc_feature 1..28: note = DNA
  misc_feature 29: note = RNA
  misc_feature 30..53: note = DNA
  source 1..53: mol_type = other DNA
cgtaaaaagaaggttttacaagactcacgttaacaatattgcagcagtacgca

SEQ ID NO: 19 - DNA, 26 bp, synthetic construct
  misc_feature 1..12: note = DNA
  misc_feature 13: note = RNA
  misc_feature 14..26: note = DNA
  source 1..26: mol_type = other DNA
  modified_base 1: mod_base = OTHER; note = a modified by HEX reporter dye
  modified_base 26: mod_base = OTHER; note = g modified by BHQ
acactagccatccttactgcgcttcg

(19) United States
(12) Patent Application Publication
(10) Pub. No.: US 2025/0092448 A1
(43) Pub. Date: Mar. 20, 2025

(54) METHODS AND COMPOSITIONS FOR NUCLEIC ACID AMPLIFICATIONS

(71) Applicants: Shoulian Dong, Mountain View, CA (US); Christopher X Dong, Mountain View, CA (US); Wiliam X Dong, Mountain View, CA (US)

(72) Inventors: Shoulian Dong, Mountain View, CA (US); Christopher X Dong, Mountain View, CA (US); Wiliam X Dong, Mountain View, CA (US)

(21) Appl. No.: 18/728,200

(22) PCT Filed: Jan. 12, 2023
(86) PCT No.: PCT/US2023/060598

§ 371 Date: Jul. 11, 2024

Related U.S. Application Data
(60) Provisional application No. 63/298,890, filed on Jan. 12, 2022.

(51) Int. Cl. C12Q 1/6844; C12Q 1/6876

(52) U.S. Cl. CPC C12Q 1/6844; C12Q 1/6876; C12Q 2600/156

(57) ABSTRACT

Described herein are methods and compositions that provide highly efficient nucleic acid amplification. In some embodiments, DNA oligonucleotide containing ribonucleotide is used as primer in the presence of RNase H2. In PCR reactions, this allows a greater than 2-fold increase of amplification product for each amplification cycle and therefore increased sensitivity and speed over conventional PCR. PCR product thus generated is used for convenient cloning. In isothermal reactions, this reduces the time needed for target detection. In some embodiment, probe containing ribonucleotide is used to monitor amplification reaction without relying on 5′-nuclease activity of DNA polymerase. The method and composition enable easy and fast detection of nucleic acid targets, enabling quick detection of genetic variations and pathogens such as SARS-CoV-2.

### FIELD

The methods and compositions described herein relate generally to the area of nucleic acid amplification and detection. In particular, described herein are methods and compositions for increasing amplification efficiency.

### BACKGROUND

A wide variety of nucleic acid amplification methods are available, and many have been employed in the implementation of sensitive diagnostic assays based on nucleic acid detection.

Polymerase chain reaction (PCR) remains the most widely used DNA amplification and quantitation method. However, PCR in general has several limitations. PCR amplification can only achieve less than two-fold increase of the amount of target sequence at each cycle. It is still relatively slow, and the sensitivity of PCR is limited. Similarly, isothermal amplification methods rely on the extension of DNA primers. Once extended, the primer is not further used to initiate another round of extension.

Primers are widely used in the various methods for nucleic acid amplification. They are generally DNA oligonucleotide designed to hybridize to target nucleic acid and extended by a polymerase. Once a primer is extended, it is incorporated into the extended amplicon and no longer used for amplification. The single-use of the standard primers place a cap on the efficiency of PCR and limits the rate of nucleic acid amplification and detection.

PCR primers for use in PCR amplification are typically all deoxynucleotides because DNA polymerases used in PCR typically do not use ribonucleotide as template. Even some polymerases are potentially capable of copying ribonucleotides (e.g., reverse transcriptase activity), the commonly held belief that substitution of a ribonucleotide for a deoxynucleotide typically increases primer cost, reduces primer stability and PCR efficiency leads to the avoidance of ribonucleotides in primers.

Efforts in increasing nucleic acid amplification efficiency have been focused on improving primer designs, polymerase functions, reaction conditions. For example, U.S. Pat. No. 8,252,558 teaches a form of nested PCR. US Patent Publication 2019/0203266A1 discloses methods to increase PCR efficiency through primer design and use of strand displacement DNA polymerase.

### SUMMARY

Described herein are methods, compositions and systems based on the use of primers that contain at least one ribonucleotide. They are designed to initiate a chain extension based on a template more than once in the presence of enzymes such as RNase H2 and polymerases.

In an aspect of the present disclosure, a nucleic acid primer set is provided. The primer set includes a first primer having a 3′ end and a 5′ end for amplifying a target nucleic acid in a sample, wherein the target nucleic acid includes a first template strand. The first primer comprises a first segment of DNA oligonucleotide and a second segment of DNA oligonucleotide, the first segment being at 5′ side of the second segment, the first segment and the second segment being linked by at least one first ribonucleotide. At least a portion of the second segment is capable of specifically hybridizing to the first template strand to initiate a nucleotide polymerization to produce an extension product complementary to the first template strand.

In some embodiments, the at least one first ribonucleotide of the first primer includes two or more ribonucleotides connected consecutively. In some embodiments, the at least one first ribonucleotide of the first primer includes two or more ribonucleotides that are spaced apart. In some embodiments, the at least one first ribonucleotide of the first primer consists of a single ribonucleotide.

In some embodiments, both the first segment and the second segment of the first primer specifically hybridize to the first template strand at a given condition. In some embodiments, the first segment does not specifically hybridize to the first template strand while the second segment specifically hybridizes to the first template strand. In some embodiments, only a portion of the second segment (and not the whole second segment) specifically hybridizes to the first template strand.

In some embodiments, the target nucleic acid further includes a second template strand complementary to the first template strand, and the primer set further comprises a second primer capable of specifically hybridizing to the second template strand. In certain embodiments, the second primer has a 3′ end and a 5′ end and comprises a third segment of DNA oligonucleotide and a fourth segment of DNA oligonucleotide, the third segment being at 5′ side of the fourth segment, the third segment and the fourth segment being linked by at least one second ribonucleotide, at least the fourth segment is capable of specifically hybridizing to the second template strand.

In another aspect of the present disclosure, a method for amplifying a target nucleic acid in a sample is provided, wherein the target nucleic acid includes a first template strand. The method includes: (a) contacting the sample with a first primer having a 3′ end and a 5′ end and comprising a first oligonucleotide segment and a second oligonucleotide segment, the first oligonucleotide segment being at 5′ side of and linked with the second oligonucleotide segment by at least one first ribonucleotide, under conditions where at least a portion of the second segment anneals to the first template strand; (b) producing a first amplicon complementary to the first template strand by extending the first primer using the first template strand as the template; and (c) cleaving the first primer at the position immediately 5′ to the at least one first ribonucleotide. The cleaving can be performed by using RNase H2.

In some embodiments, the first segment anneals to the first template strand under the conditions, the method further comprising producing a second amplicon complementary to the first template strand by extending the first segment of the first primer using the first template strand as a template while replacing the first amplicon from the first template strand.

In some embodiments, wherein the first segment of the first primer does not anneal to the first template strand under the conditions, the method further comprising: prior to cleaving at (c): separating the first amplicon from the first template strand; using a second primer to produce a nucleic acid strand complementary to the first amplicon, the new nucleic acid strand including a segment complementary to the first segment of the first primer; and after cleaving at (c): producing a third amplicon complementary to the new nucleic acid strand by extending the first segment of the first primer using the new nucleic acid strand as a template, while replacing the first amplicon from the new nucleic acid strand. In some of these embodiments, wherein at least a portion to 3′ side of the at least one first ribonucleotide and distal to the 3′ end of the first primer does not specifically hybridize to the first template strand.

In some embodiments, the target nucleic acid further includes a second template strand complementary to the first template strand, the method further comprises: (d) contacting the sample with a second primer comprising a third segment of DNA oligonucleotides and a fourth segment of DNA oligonucleotides, the third segment being at the 5′ side of and linked with the fourth segment by at least one second ribonucleotide, where at least a portion of the fourth segment anneals to the second template strand under the conditions; (e) producing a second amplicon complementary to the second template strand by extending the second primer using the second template strand as the template; and (f) cleaving the second primer at the position immediately 5′ to the at least one second ribonucleotide.

In some embodiments, the first segment and second segment both anneal to the first template strand under the conditions, the target nucleic acid further includes a second template strand complementary to the first template strand, the method further comprises: (d) contacting the sample with a second primer comprising a third segment of DNA oligonucleotides and a fourth segment of DNA oligonucleotides, the third segment being at the 5′ side of and linked with the fourth segment by at least one second ribonucleotide, where both the third segment and fourth segment anneal to the second template strand under the conditions; (e) producing a second amplicon complementary to the second template strand by extending the second primer using the second template strand as the template; (f) cleaving the second primer at the position immediately 5′ to the at least one second ribonucleotide; and (g) producing a third amplicon complementary to the first template strand by extending the first segment of the first primer using the first template strand as a template while replacing the first amplicon from the first template strand, and producing a fourth amplicon complementary to the second template strand by extending the second primer using the second template strand as the template while replacing the third amplicon from the second template strand. In some of these embodiments, the method can further comprise: (h) producing a fifth amplicon complementary to the first amplicon using another second primer; and producing a sixth amplicon complementary to the second amplicon using another first primer. Thermocycling can be performed to further amplify the amplification products obtained in the process, e.g., increasing temperature to separate double-stranded amplicons produced; and decreasing temperature to anneal further first primers and second primers to the separated amplicons.

In some embodiments of the methods described herein, the first primer is anchored on a solid surface or in a matrix.

In some embodiments of the methods described herein, wherein the first template strand is an RNA.

In some embodiments, the first segment cleaved from the first primer is used as a primer to initiate nucleotide polymerization using a second target nucleic acid as a template.

In a further aspect, method of detecting a nucleotide variation in a target nucleic acid in a sample is provided. The sample contains a first double-stranded DNA and a second double stranded DNA, the second double stranded DNA differing from the first double stranded DNA at at least one variance position. The method comprises:
(a) contacting the sample with a first primer having a 3′ end and a 5′ end and comprising a first oligonucleotide segment and a second oligonucleotide segment, the first oligonucleotide segment being at 5′ side of and linked with the second oligonucleotide segment by at least one first ribonucleotide, under conditions such that the first primer overall anneals to a first template strand of the first double-stranded DNA as well as the first template strand of the second double-stranded DNA, but the second segment of the first primer by itself does not anneal to the first template strand of the first double-stranded DNA or to the first template strand of the second double-stranded DNA, wherein the ribonucleotide of the first primer is aligned with or in the vicinity of the variance position such that the first primer is perfectly matched with the first template strand of the first double-stranded DNA but not perfectly matched with the first template strand of the second double-stranded DNA;
(b) producing a first amplicon by extending the first primer hybridized on the first template of the first double-stranded DNA using the first template strand of the first double-stranded DNA as the template, and producing a second amplicon by extending the first primer hybridized on the first template of the second double-stranded DNA using the first template strand of the second double-stranded DNA as the template;
(c) cleaving the first primer hybridized with the first template strand of the first double-stranded DNA at the position immediately 5′ to the at least one first ribonucleotide using an RNase to produce 5′ side segment cleaved from the first primer hybridized with the first template strand of the first double-stranded DNA, while not cleaving the first primer hybridized with the first template strand of the second double-stranded DNA; and
(d) conducting PCR to further amplify the first and second double-stranded DNA in the presence of the RNase to thereby differentiate the first and second double-stranded DNA by a quantity of respective amplicon products.

In a further aspect, a method for amplifying a target nucleic acid in a sample is provided, wherein the target nucleic acid includes a first template strand. The method comprises:
(a) contacting the sample with a first primer having a 3′ end and a 5′ end and comprising a first oligonucleotide segment and a second oligonucleotide segment, the first oligonucleotide segment being at 5′ side of and linked with the second oligonucleotide segment by at least one first ribonucleotide, under conditions where at least a portion of the second segment anneals to the first template strand;
(b) producing a first extension product complementary to the first template strand by extending the first primer using the first template strand as the template; and
(c) conducting PCR to produce duplicate copies of the first extension product, each of the duplicate copies containing a first primer;
(d) cleaving the first primers in the amplicons of the first extension product at the position immediately 5′ to the at least one first ribonucleotide to produce cleaved portions of the duplicate copies; and
(e) annealing or ligating the cleaved portions of the duplicate copies with other nucleic acid strands.

In a further aspect, a molecular probe for detecting amplification of a nucleic acid is provided. The probe comprises a quencher portion, a fluorophore portion, and a linker portion linking the quencher portion and the fluorophore portion, the linker portion comprising a plurality of deoxyribonucleotides and a ribonucleotide, wherein no or low fluorescence is given by the fluorophore when then linker is intact. Accordingly, a method of detecting an amplicon product in an amplification system is provided, the method includes allowing such a molecular probe to specifically hybridize to an amplicon product in an amplification system; cleaving the ribonucleotide in the molecular probe; and detecting fluorescence given off by the fluorophore portion of the molecular probe.

Various embodiments contemplated herein may include, but need not be limited to, one or more of the following.

### BRIEF DESCRIPTION OF THE DRAWINGS

FIG. 1 is a schematic drawing showing a ribonucleotide-containing primer amplification of a template and afterward being cleaved by RNase H2 and ready for second round of extension, according to embodiments of the present invention.

FIG. 2 is a schematic drawing showing the extension and displacement of an amplicon using a primer containing two spaced-apart ribonucleotides in the presence of a strand-displacement DNA polymerase and an RNase H2, according to embodiments of the present invention.

FIG. 3 is a schematic drawing showing the extension and displacement of an amplicon using a primer containing multiple consecutive ribonucleotides in the presence of a strand-displacement DNA polymerase and an RNase, according to embodiments of the present invention.

FIG. 4 is a schematic drawing showing the generation of a DNA template through reverse transcription of an RNA template with a ribonucleotide containing primer in the presence of a reverse transcriptase with RNase H activity or a reverse transcriptase plus an RNase H, according to embodiments of the present invention.

FIG. 5 is a schematic drawing showing the amplification of whole genome or whole transcriptome or a plural of target DNA by the use of multiple primers each containing a ribonucleotide, where the primers anneal to template and are extended by a polymerase and then cleaved by a RNase H2 and further extended by a polymerase, according to embodiments of the present invention.

FIG. 6 is a schematic drawing showing the amplification of whole genome or whole transcriptome or a plural of target DNA by the use of multiple primers in the presence of NTPs, where ribonucleotides are incorporated by a polymerase and is then cleaved by a RNase H2 and further extended by a polymerase, and the primers optionally contain at least a ribonucleotide, according to embodiments of the present invention.

FIG. 7 is a schematic drawing showing the generation of two amplicons from one ribonucleotide containing primer that includes a tag sequence that is not substantially matched to the target template, according to embodiments of the present invention.

FIG. 8 is a schematic drawing showing the generation of two amplicons from one ribonucleotide containing primer to incorporate sequences that facilitate downstream amplification, according to embodiments of the present invention.

FIG. 9 is a schematic drawing showing amplification of templates with two ribonucleotide containing primers, according to embodiments of the present invention.

FIG. 10 is a schematic drawing showing a single tube isothermal amplification of RNA by combination of ribonucleotide primer-initiated amplification with RNA polymerase, according to embodiments of the present invention.

FIGS. 11a and 11b are schematic drawings showing the initiation and amplification cascade using ribonucleotide containing loop primers, according to embodiments of the present invention.

FIG. 12 is a schematic drawing showing strand displacement chain reaction, according to embodiments of the present invention.

FIG. 13 is a schematic drawing showing the amplification of DNA template using ribonucleotide containing primers by a non-strand displacement DNA polymerase, according to embodiments of the present invention. The amplicons have recessive 5′ ends that are generated by the cleavage of ribonucleotide primers. The 3′-overhangs are determined by the placement of the ribonucleotide and the 5′ side sequences. When the overhangs are design to be complementary, the amplicon forms circular structure by annealing of the single-stranded 3′ ends. The circular DNA is further transfected into recipient cells.

FIG. 14 is a schematic drawing showing target amplification and direct cloning of amplicons generated by ribonucleotide containing primers into a vector, according to embodiments of the present invention. Amplicons generated using ribonucleotide containing primers by a non-strand displacement DNA polymerase have recessive 5′ end due to RNase H2 cleavage at the 5′ side of the ribonucleotide. The overhangs are designed to anneal with a pre-selected vector. The mixed amplicons and vectors form circular structure and are ready to be transfected into recipient cells.

FIG. 15 is a schematic drawing showing amplicon concatemerization using ribonucleotide-containing primers, according to embodiments of the present invention.

FIG. 16 shows genotyping assays using a ribonucleotide containing primer according to embodiments of the present invention. A ribonucleotide incorporated at or near the variation site is not cleavage by RNase H2 when mismatched to the variation but cleaved when matches the target. The primer is so designed to have short 3′ segment that once cleaved and copied the 3′ segment doesn't form stable hybridization and the primer fails to anneal in following round of amplification. A ribonucleotide designed to match the wild type sequence does not amplify the wild type sequence but amplify any mutant efficiently.

FIG. 17 shows a haplotyping assay using ribonucleotide containing primers according to embodiments of the present invention. A haplotype is amplified efficiently only when both of the targeted variable sites are mismatched at or near the ribonucleotide.

FIG. 18 shows initiation of amplification cascade using ribonucleotide containing primers according to embodiments of the present invention. Amplification of a third target is designed to initiate only upon the amplification of the other two targets and the ensued cleavage at the nucleotides. The cleaved 5′ segments of two of the primers serve as primers to amplify the third target.

FIG. 19 shows sequential initiation of amplification cascade using ribonucleotide containing primer for multiplex target detection, according to embodiments of the present invention.

FIG. 20 shows initiation of amplification cascade using ribonucleotide containing primer for target proportion imbalance detection, according to embodiments of the present invention. Cleaved 5′ segments to the ribonucleotide in the first two amplicons are designed to anneal at 1:1 ratio and extend each other. Once extended, the 5′ segments fail to prime the amplification of a third target. Imbalance in the starting template amount leads to accumulation of excessive amount of a 5′ segment of one primer, which is used to amplify a third target.

FIG. 21 is a schematic drawing showing an example of the amplification of a target by anchored primers, according to embodiments of the present invention.

FIG. 22 is a schematic drawing showing bridge amplification using ribonucleotide containing primers according to embodiments of the present invention.

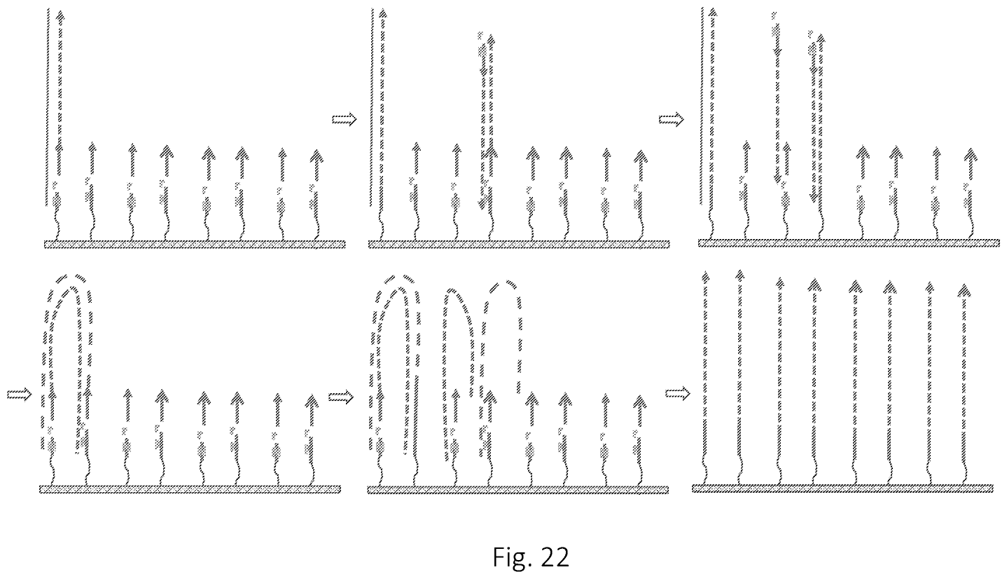

FIG. 23 is a schematic drawing showing signal detection of ribonucleotide-containing probes according to embodiments of the present invention.

FIG. 24 shows the amplification curve of R-primer amplifying a target in comparison with standard primers showing similar amplification Ct in the absence of RNase H2 but higher Ct in the presence of RNase H2.

FIG. 25 shows comparison of melting curve of amplicons generated with R-Primer in the absence with that in the presence of RNase H2.

FIG. 26 shows specific detection of EGFR Exon21 cancer mutation with R-primer and non-SD polymerase.

FIG. 27 shows the acceleration of target detection by using ribonucleotide containing primers with RNase H2 in isothermal LAMP assays.

FIG. 28 shows the earlier detection of fluorescence intensity increase in isothermal reaction using R-Primers than standard primers.

FIG. 29 shows the comparison of the amplification by Strand Displacement Chain Reaction (SDCR) to standard PCR.

FIG. 30 shows fluorescence increase of a ribo-probe detected during target amplification.

FIG. 31 shows earlier detection of SARS-CoV-2 gene by real time SDCR than PCR with standard primers.

FIG. 32 shows more-than-two-fold per cycle amplification of SDCR with ribo-primers.

### DETAILED DESCRIPTION

### Definitions

Terms used in the claims and specification are defined as set forth below unless otherwise specified.

The term “nucleic acid” refers to a nucleotide polymer, and unless otherwise limited, includes analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e., a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to the capacity for precise pairing between two nucleotides; i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Specific hybridization” or “specifically hydridizing” refers to the binding of a nucleic acid to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference for their descriptions of stringent hybridization conditions). As indicated by standard references, a simple estimate of the Tₘ value may be calculated by the equation: Tₘ=81.5+0.41 (% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonucleotide sequences based on nearest-neighbors thermodynamics can carried out as described in “A unified view of polymer, dumbbell, and oligonucleotide DNA nearest-neighbor thermodynamics” John Santa Lucia, Jr., PNAS Feb. 17, 1998 vol. 95 no. 4 1460-1465 (which is incorporated by reference herein for this description).

The term “oligonucleotide” refers to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “DNA oligonucleotide” refers to an oligonucleotide whose components are all DNA or DNA analogues that are not RNA, for example, DNA derivatives including modified bases (such as methylated, hydroxymethylated, halo-substituted bases), LNA, etc.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer or segments thereof depends on the intended use of the primer, but primers are typically at least 7 nucleotides long and, in some embodiments, range from 10 to 30 nucleotides, or, in some embodiments, from 10 to 60 nucleotides, in length. In some embodiments, primers can be, e.g., 15 to 50 nucleotides long. Short primer molecules generally require cooler temperatures to form sufficiently stable hybrid complexes with the template. A primer need not reflect the exact sequence of the template but must be sufficiently complementary to hybridize with a template.

A primer is said to anneal to another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence.” This description encompasses primers that anneal wholly to the nucleotide sequence, as well as primers that anneal partially to the nucleotide sequence.

The term “ribonucleotide containing primer” (also referred to herein as “Ribo-Primer” or “R-Primer”) refers to a primer that includes one or more ribonucleotides. The “ribonucleotide containing primer” anneals with a nucleic acid and initiate nucleotide polymerization. The 3′ end of the primer can have 3′-hydroxyl group or be blocked but activatable. The ribonucleotide in the primer can be replicated by some polymerases, esp. reverse transcriptase or proof-reading DNA polymerases. When the primer is incorporated into a double strand DNA, the ribonucleotide can be recognized by a RNase H2 and cleaved at the 5′ side to form a 3′ hydroxyl upstream segment and a 5′ phosphate downstream segment. The ribonucleotide can be used to activate the primer when incorporated into a 3′-blocked primer or re-initiate the extension of the primer starting with the upstream segment.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientations in some embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable label to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size. Generally, probes are at least 7 to 15 nucleotides in length. Other probes are at least 20, 30, or 40 nucleotides long. Still other probes are somewhat longer, being at least 50, 60, 70, 80, or 90 nucleotides long. Yet other probes are longer still, and are at least 100, 150, 200 or more nucleotides long. Probes can also be of any length that is within any range bounded by any of the above values (e.g., 15-20 nucleotides in length).

The primer or probe can be perfectly complementary to the target nucleotide sequence or can be less than perfectly complementary. In some embodiments, the primer has at least 65% identity to the complement of the target nucleotide sequence over a sequence of at least 7 nucleotides, more typically over a sequence in the range of 10-30 nucleotides, and, in some embodiments, over a sequence of at least 14-25 nucleotides, and, in some embodiments, has at least 75% identity, at least 85% identity, at least 90% identity, or at least 95%, 96%, 97%, 98%, or 99% identity. It will be understood that certain bases (e.g., the 3′ base of a primer) are generally desirably perfectly complementary to corresponding bases of the target nucleotide sequence. Primer and probes typically anneal to the target sequence under stringent hybridization conditions.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include PCR, nucleic acid strand-based amplification (NASBA), two-step multiplexed amplifications, rolling circle amplification (RCA), loop mediated amplification (LAMP), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), helicase-dependent amplification (HDA), and the like. Descriptions of such techniques can be found in, among other sources, Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4 (1): 41-7, U.S. Pat. Nos. 6,027,998; 6,605,451, Barany et al., PCT Publication No. WO 97/31256; Wenz et al., PCT Publication No. WO 01/112579; Day et al., Genomics, 29 (1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27: e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13 (2): 294-307, and Landegren et al., Science 241:1077-80 (1988), Demidov, V., Expert Rev Mol Diagn. 2002 November; 2 (6): 542-8., Cook et al., J Microbiol Methods. 2003 May; 53 (2): 165-74, Schweitzer et al., Curr Opin Biotechnol. 2001 February; 12 (1): 21-7, U.S. Pat. Nos. 5,830,711, 6,027,889, 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

A “multiplex amplification reaction” is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction;” all terms refer to PCR with real-time signal detection.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation.

A DNA polymerase is said to be “stable” at a particular temperature if it provides a satisfactory extension rate in a nucleic acid amplification reaction.

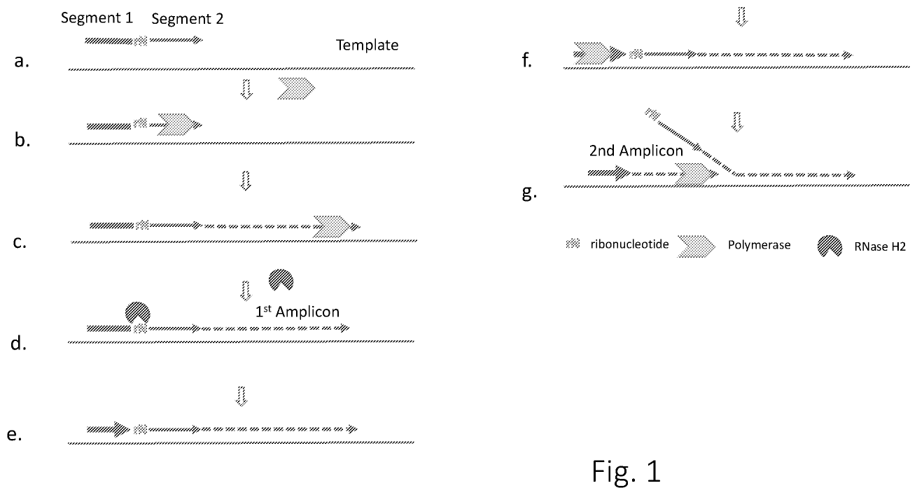

FIG. 1 shows a scheme to use ribonucleotide containing primer for DNA amplification. For ease of discussion, this primer can be considered to be a “forward” primer. The ribonucleotide gives the primer a second chance to initiate replication once cleaved at the 5′ side of the ribonucleotide.

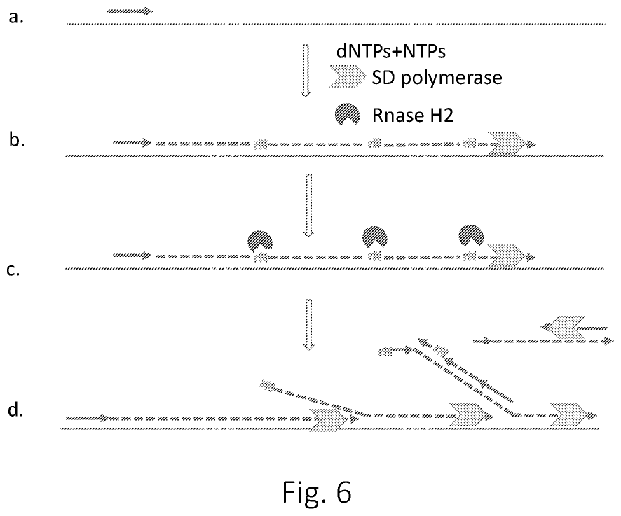

In one embodiment, the primer is designed to have two segments, the upstream segment at the 5′ end (Segment 1) and the downstream segment at the 3′ end (Segment 2), with a ribonucleotide joining them together (see FIG. 1a). The ribonucleotide also matches the target template. Each of the segments are designed to have high enough Tm to stably hybridize to the template at the reaction conditions that enable amplification. The ribonucleotide containing primer is first annealed to target template (see FIG. 1b) and extended by a polymerase to make a copy of the template (i.e., a complementary strand for the template), forming a double stranded DNA (FIG. 1, c). The ribonucleotide is recognized by a RNase H2 (FIG. 1d) and the enzyme cleaves at the 5′-side of the ribonucleotide and produces a 3′-hydroxyl terminus for Segment 1 (FIG. 1e) and 5′-phosphate ribonucleotide terminus for Segment 2.

The disclosed methods make the use of a RNase H to cleave the ribonucleotide containing primer. When there is a single ribonucleotide surrounded by DNA sequences, RNase H2 is used due to its specificity for single ribonucleotide recognition and cleavage. A continuous stretch of ribonucleotides equal or more than two can also be used within the ribonucleotide containing primer, in which case RNase H is preferred to digest the RNA and make Segment 1 extendable.

Ribonuclease H is a family of non-sequence-specific endonuclease enzymes that catalyze the cleavage of RNA in an RNA/DNA substrate via a hydrolytic mechanism. RNase H2, aka. RNase H II, is a member of RNase H family. The enzyme binds to RNA-DNA duplexes and nicks 5′ to a ribonucleotide. It specifically cleaves at the ribonucleotide in an RNA-DNA hybrid. It does not hydrolyze the phosphodiester bonds when the sequence is single stranded or the ribonucleotide is mismatched. Upon cleavage, the enzyme leaves a 5′ phosphate end for the downstream segment and a 3′ hydroxyl end for the upstream segment. RNase H2 from different sources has different enzymatic activity. RNase H2 cloned from E. coli is available from New England Biolab (NEB) with optimal activity at 37° C. A thermostable RNase H2, cloned from Pyrococcus abyssi, is available from Integrated DNA Technologies (IDT). It has high activity at 70-95° C. but low activity below 50° C. In some embodiments, E. coli RNase H2 is included in the reaction. In some embodiments, RNase H2 active at low temperature is used in the reaction. If thermocycling or high temperature incubation is to be carried out (as in PCR and isothermal amplifications), the RNase H2 is preferably thermostable.

In some embodiments, the polymerase has strand displacement activity (FIG. 1g). The ribonucleotide containing primer is first extended to generate a copy of target template. The ribonucleotide is recognized and cleaved by a RNase H2 enzyme to generate an extendable upstream Segment 1. Segment 1 is then extended from the 3′-hydroxyl group by the strand-displacement polymerase activity to generate a second copy of the target template and in the meantime displaces the initially generated copy.

When the polymerase lacks strand displacement activity, Segment 1 does not displace Segment 2 and its extension product. However, Segment 1 can be designed to be used as a primer by itself once cleaved by the RNase.

In some embodiments, the position of the ribonucleotide within the primer is adjusted to make Segment 1 and Segment 2 to have different Tm. In one further embodiment, Segment 2 can be made short to have a Tm that is too low to hybridize to the template strand by itself.

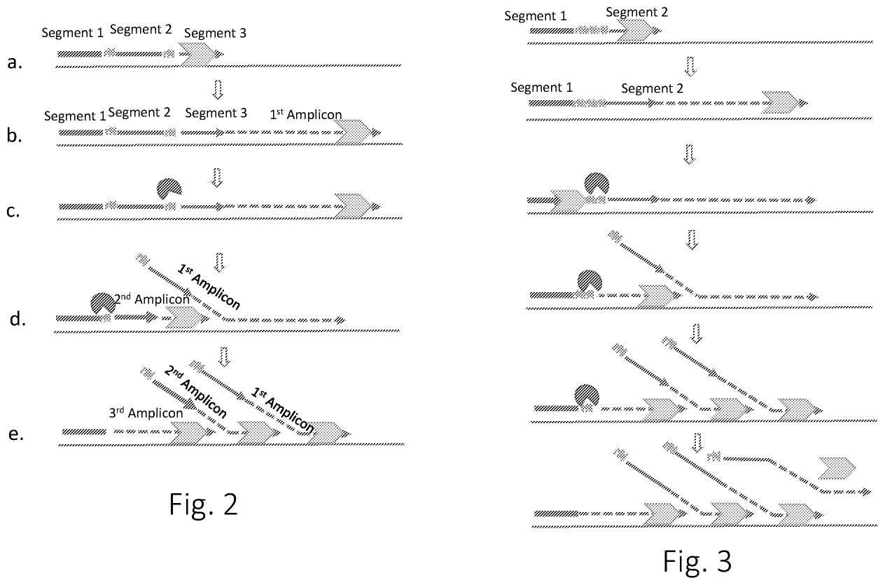

In a further embodiment, more than one ribonucleotide is included in the primer. For example, when two ribonucleotides are included in the primer, a third segment can be further included in the primer. After cleavage, one primer generates up to three amplicons (FIG. 2).

In another embodiment, Segment 1 is partially complementary to the template. As an example, Segment 1 has a portion on its 3′ end that is complementary to the template. The other portion can be any sequences of choices.

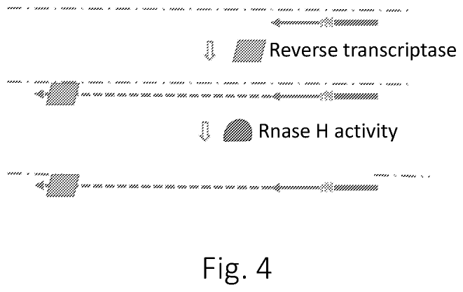

In some embodiments, more than one ribonucleotide are included in a R-primer. In one embodiment, the ribonucleotides are placed inside the primer, separated by deoxynucleotides (FIG. 2). Three segments can be separated by the two ribonucleotides (FIG. 2a). The primer is extended by polymerase from Segment 3 (FIG. 2, b) and one of ribonucleotide is then recognized by RNase H2 and cleaved at the 5′ side of the ribonucleotide, making Segment 2 extendable (FIG. 2, c). The extending Segment 2 displaces Segment 3 and the first amplicon of the template formed by Segment 3 extension. The second ribonucleotide can also be recognized by RNase H2 and cleaved at its 5′ side, making Segment 1 extendable (FIG. 2, d). Extension of Segment 1 with a polymerase with strand displacement activity leads to the displacement of Segment 2 and second amplicon generated by the extension of Segment 2. As a result, one primer leads to up to three amplicons. In another embodiment, the ribonucleotides are placed side-by-side within the primer (FIG. 3). Each ribonucleotide can initiate extension of from the 3′-hydroxyl terminus of the segment at its 5′-side when cleaved by a RNase. More than one copies of target template can be generated from one primer. RNase H can also be used to cleave the stretch of RNA and make Segment 2 extendable to produce the second amplicon. Other ribonucleotide structures can be designed to fit with the specificities of RNase of choice or the engineered RNase.

RNases have variable activities and stabilities at different temperature. For isothermal amplification at low temperature, an RNase H or RNase H2 with optimal activity and stability at the targeted temperature can be used. When the amplification is carried out at elevated temperature an RNase with high activity at the targeted temperatures is needed. For amplification through thermocycling, a thermostable RNase H or RNase H2 with high activity at the extension temperature is required.

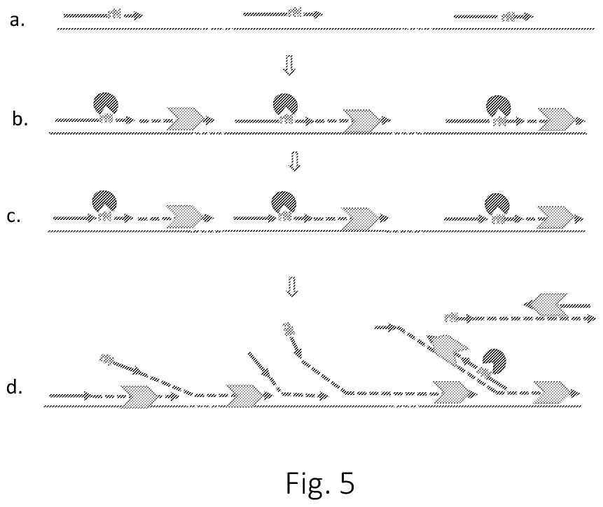

In some embodiments, RNA template is used with ribonucleotide containing primers. In FIG. 4, RNA template is reverse transcribed by a reverse transcription reaction by a ribonucleotide containing primer. The cDNA strand thus generated contains the ribonucleotide in its sequence.

### Samples

Nucleic acid template used in the invention can DNA or RNA or chimeric DNA and RNA. Nucleic acid-containing samples can be obtained from biological sources and prepared using conventional methods known in the art. In particular, nucleic acid useful in the methods described herein can be obtained from any source, including unicellular organisms and higher organisms such as plants or non-human animals, e.g., canines, felines, equines, primates, and other non-human mammals, as well as humans. In some embodiments, samples may be obtained from an individual suspected of being, or known to be, infected with a pathogen, an individual suspected of having, or known to have, a disease, such as cancer, or a pregnant individual.

Nucleic acids can be obtained from cells, bodily fluids (e.g., blood, a blood fraction, urine, etc.), or tissue samples by any of a variety of standard techniques. In some embodiments, the method employs samples of plasma, serum, spinal fluid, lymph fluid, peritoneal fluid, pleural fluid, oral fluid, and external sections of the skin; samples from the respiratory, intestinal genital, or urinary tracts; samples of tears, saliva, blood cells, stem cells, or tumors. Samples can be obtained from live or dead organisms or from in vitro cultures. Illustrative samples can include single cells, paraffin-embedded tissue samples, and needle biopsies. In some embodiments, the nucleic acids analyzed are RNA (FIG. 4). The RNA may be converted to DNA by reverse transcriptase to form RNA: DNA heteroduplex. The DNA may be made into single-stranded by digestion of RNA by a RNase H or RNase H activity of reverse transcriptase.

Nucleic acids of interest can be isolated using methods well known in the art. The sample nucleic acids need not be in pure form, but are typically sufficiently pure to allow the steps of the methods described herein to be performed.

### Target Nucleic Acids

Any target nucleic acid that can detected by nucleic acid amplification can be detected using the methods described herein. In typical embodiments, at least some nucleotide sequence information will be known for the target nucleic acids. For example, if the amplification reaction employed is PCR, sufficient sequence information is generally available for each end of a given target nucleic acid to permit design of suitable amplification primers.

The targets can include, for example, nucleic acids associated with pathogens, such as viruses, bacteria, protozoa, or fungi; RNAs, e.g., those for which over- or under-expression is indicative of disease, those that are expressed in a tissue- or developmental-specific manner; or those that are induced by particular stimuli; genomic DNA, which can be analyzed for specific polymorphisms (such as SNPs), alleles, or haplotypes, e.g., in genotyping. Of particular interest are genomic DNAs that are altered (e.g., amplified, deleted, and/or mutated) in genetic diseases or other pathologies; sequences that are associated with desirable or undesirable traits; and/or sequences that uniquely identify an individual (e.g., in forensic or paternity determinations).

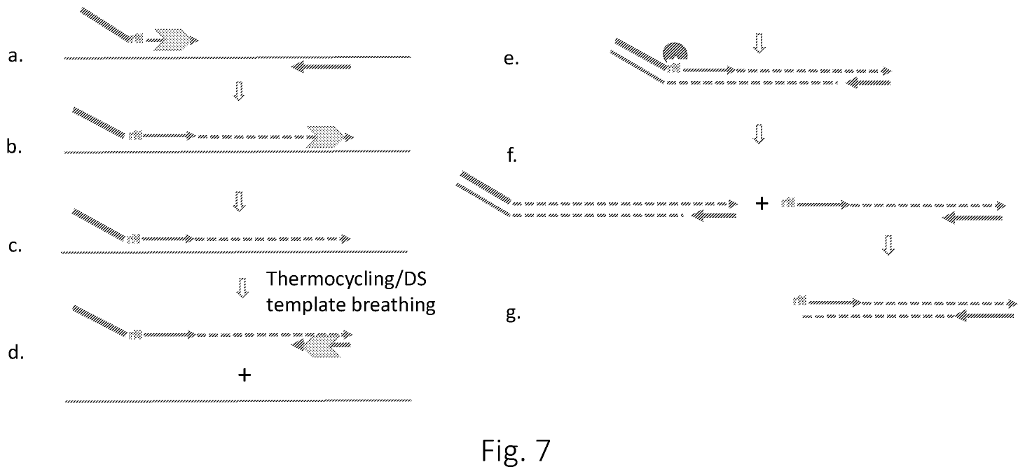

In some embodiments, the targets contain the total of DNA from an organism (FIGS. 5 & 6). In another embodiment, the targets contain the total of cDNA from RNA. In preferred embodiments, the total of DNA or cDNA may be amplified using ribonucleotide containing degenerate primers in the presence of RNase H2 (FIG. 5, a). A ribonucleotide is incorporated into degenerate primers and the primers can anneal across the target genome or transcriptome. The ribonucleotide is preferentially incorporated at the position 3 bases away from the 3′ end. Each annealed random primer is extended by a strand displacement polymerase (FIG. 5, b). One suitable polymerase is phi29 DNA polymerase. The ribonucleotide in each extended primer is then recognized and cleaved at the 5′ side of the base (FIG. 5, c), and further extended from the 5′ segment of the primer (FIG. 5, d). The extending 5′ segment of each primer displaces the 3′ segment and the first amplicon from the 3′ end of the primer. The displaced amplicons then serve as templates, and anneal with primers with complementary sequences and are amplified further. The ribonucleotide containing degenerate primers are expected to generate higher yield of whole-genome amplified targets. In another embodiment, the total of DNA or cDNA may be amplified using degenerate primers in the presence of ribonucleotide triphosphates and RNase H2 (FIG. 6; a). With a polymerase that can incorporate NTPs, each annealed primer is extended by the polymerase. Ribonucleotides are incorporated into the extending sequence randomly (FIG. 6, b). The newly incorporated ribonucleotides are then recognized and cleaved at the 5′ side (FIG. 6, c). The sequences on the 3′ side of each ribonucleotide are then further extended and displaces the sequences in the front of the growing amplicons (FIG. 6, d). In a similar embodiment, a locus-specific primer is used in place of the degenerate primers in the presence of NTPs and RNase H2 to amplify target nucleic acids with polymerase that incorporates ribonucleotides. In some embodiments, the ribonucleotide containing primer or primers are combined with rolling circle amplification (RCA).

### Primer Design

Primers suitable for nucleic acid amplification are sufficiently long to prime the synthesis of extension products in the presence of a suitable nucleic acid polymerase. The exact length and composition of the primer will depend on many factors, including, for example, temperature of the annealing reaction, source and composition of the primer, and where a probe is employed, proximity of the probe annealing site to the primer annealing site and ratio of primer:probe concentration. For example, depending on the complexity of the target nucleic acid sequence, an oligonucleotide primer typically contains in the range of about 10 to about 60 nucleotides, although it may contain more or fewer nucleotides. The primers should be sufficiently complementary to selectively anneal to their respective strands and form stable duplexes.

In general, one skilled in the art knows how to design suitable primers capable of amplifying a target nucleic acid of interest. For example, PCR primers can be designed by using any commercially available software or open source software, such as Primer3 (see, e.g., Rozen and Skaletsky (2000) Meth. Mol. Biol., 132:365-386; www.broad.mit.edu/node/1060, and the like) or by accessing the Roche UPL website. The amplicon sequences are input into the Primer3 program with the UPL probe sequences in brackets to ensure that the Primer3 program will design primers on either side of the bracketed probe sequence.

Primers may be prepared by any suitable method, including, for example, direct chemical synthesis by methods such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetra. Lett., 22:1859-1862; the solid support method of U.S. Pat. No. 4,458,066 and the like, or can be provided from a commercial source. Primers may be purified by using a Sephadex column (Amersham Biosciences, Inc., Piscataway, N.J.) or other methods known to those skilled in the art. Primer purification may improve the sensitivity of the methods described herein.

Ribonucleotide containing primers (shown in FIG. 1) include one or more ribonucleotides in the sequence and segments of DNA oligonucleotides. They may be made by any suitable methods, including, for example, direct chemical synthesis; or can be provided from a commercial source.

“Stabilizing bases” include, e.g., stretches of peptide nucleic acids (PNAs) that can be incorporated into DNA oligonucleotides to increase duplex stability. Locked nucleic acids (LNAs) and unlocked nucleic acids (UNAs) are analogues of RNA that can be easily incorporated into DNA oligonucleotides during solid-phase oligonucleotide synthesis, and respectively increase and decrease duplex stability. Suitable stabilizing bases also include modified DNA bases that increase the stability of base pairs (and therefore the duplex as a whole). These modified bases can be incorporated into oligonucleotides during solid-phase synthesis and offer a more predictable method of increasing DNA duplex stability. Examples include AP-dC (G-clamp) and 2-aminoadenine, as well as 5-methylcytosine and C(5)-propynylcytosine (replacing cytosine), and C(5)-propynyluracil (replacing thymine).

“Destabilizing bases” are those that destabilize double-stranded DNA by virtue of forming less stable base pairs than the typical A-T and/or G-C base pairs. Inosine (I) is a destabilizing base because it pairs with cytosine (C), but an I-C base pair is less stable than a G-C base pair. This lower stability results from the fact that inosine is a purine that can make only two hydrogen bonds, compared to the three hydrogen bonds of a G-C base pair. Other destabilizing bases are known to, or readily identified by, those of skill in the art. Ribonucleotide containing primer in a primer set

A ribonucleotide containing primer can be employed with a simple conventional reverse primer or another ribonucleotide containing primer in a reaction.

Primer with Tag Sequence

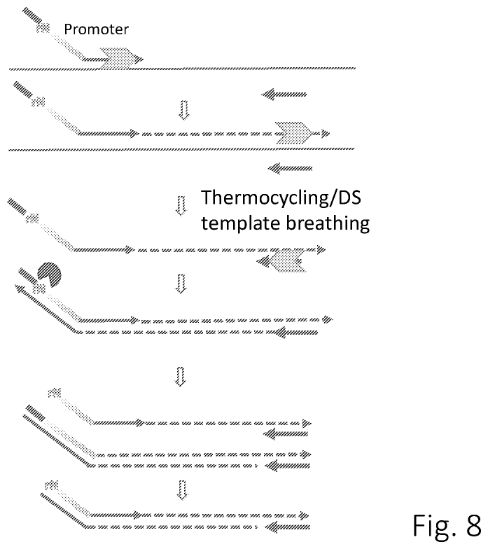

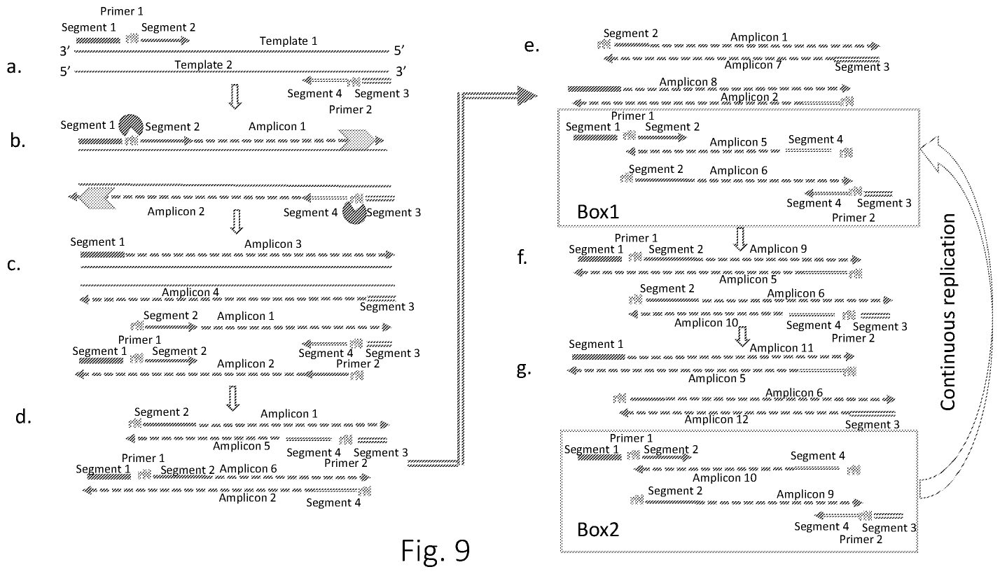

A primer may be added one or more tag sequences at the 5′-end (FIGS. 7 & 8). The 3′ segment of the primer hybridizes with target template while the 5′ segment may be partially complementary or non-complementary with the target template. The 3′ segment is designed to be locus-specific to the target. The tag may consist of nucleotides that may not hybridize with the target nucleic acid. One or more ribonucleotides may be included in the locus-specific region (FIG. 7) or in the tag region (FIG. 8). The tag can be any sequences designed to facilitate downstream reactions. In one example, the tag may be T7 promoter, M13 promoter, SP promoter and other promoters or the combination of them. In some embodiments, the tag may include a nicking restriction site for nicking enzyme assisted reaction (NEAR). In some embodiments, the tag may contain a promoter and may be used to initiate in vitro transcription of the target sequence. In some embodiments, incorporation of the tag and reactions using the tag are included in a single tube. As shown in FIG. 7, a second primer may be provided to form a primer set with the ribonucleotide containing primer. The ribonucleotide containing primer first anneals with a template (FIG. 7, a) and is extended by a polymerase (FIG. 7, b). The double stranded extension product (FIG. 7, c) can be then denatured. The denaturation can be carried out by increasing temperature or alkaline denaturation or double strand DNA breathing. The second primer, shown in FIG. 7 as the reverse primer, then anneals to the single stranded extension product of the ribonucleotide containing primer and make a copy of it by polymerase and thus generate the complementary strand of the tag segment (FIG. 7, e). The ribonucleotide in the regenerated double strand amplicon is then recognized by RNase H2 and cleaved at the 5′ side to produce an extendable tag segment. The tag segment is then extended and displace the sequences in front of it by the strand displacement activity of the polymerase (FIG. 7, f). As a result, the tag is incorporated into the amplifiable structure. Further cycles of amplification benefits from the increased efficiency through the ribonucleotide containing primer.

When the tag sequence in FIG. 7 is designed as some functional elements, such as RNA polymerase promoter or nicking enzyme recognition site (FIG. 8), amplification of the target nucleic acids can be further combined with other nucleic acid amplification methods, such as nucleic acid sequence-based amplification (NABSA) or NEAR.

### Pair of Ribonucleotide Containing Primers

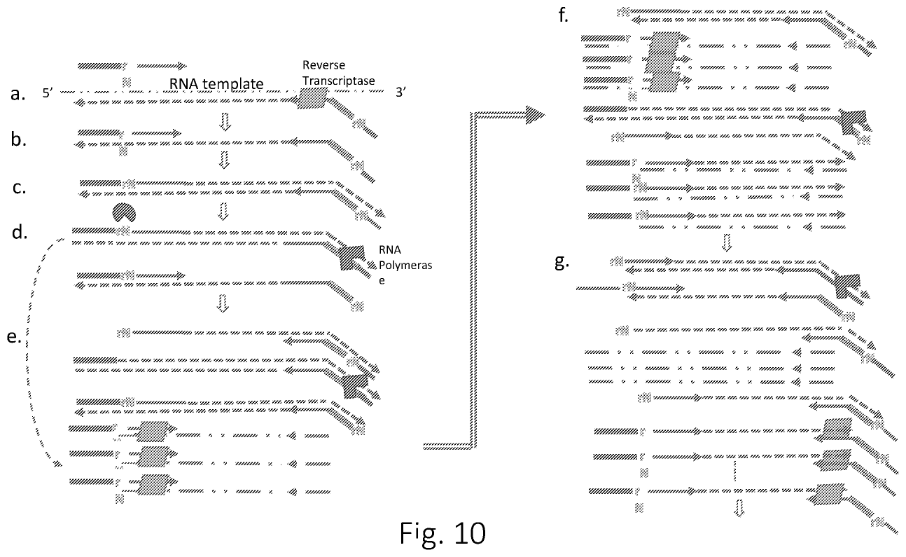

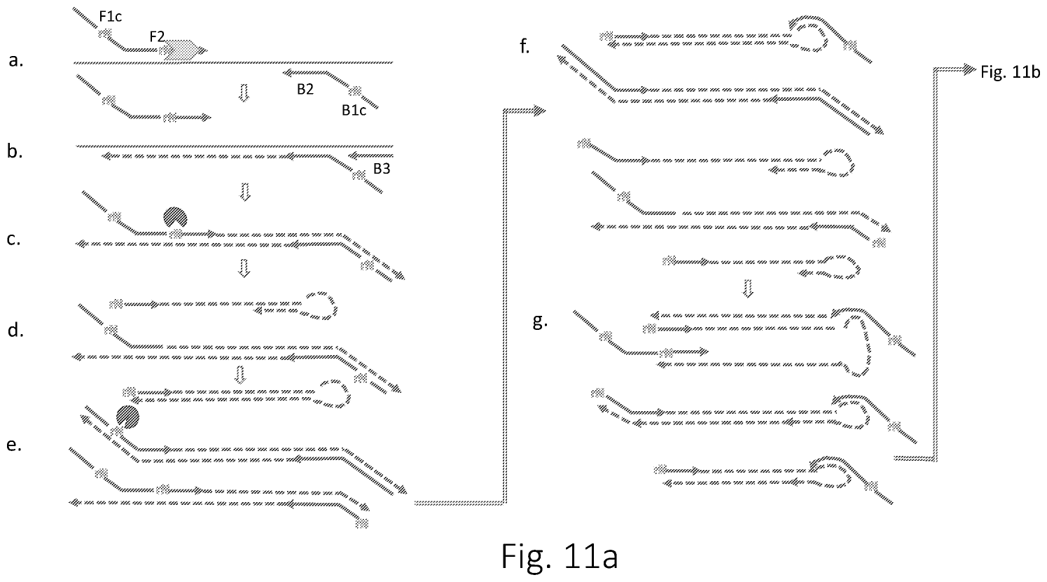

In some embodiments, both forward primer and reverse primer are ribonucleotide containing primers (FIG. 9). FIG. 9 generally describes the amplification process by using a pair of ribonucleotide containing primers with a polymerase with strand displacement activities in the presence of RNase H2. Each of the forward and reverse primer is designed with two segments separated by a nucleotide (FIG. 9, a). Primer 1, shown as forward primer, has Segment 1 at the 5′ end (thin solid line) and Segment 2 at the 3′ end (thick solid line). Primer 2, shown as reverse primer, is designed with two segments separated by a ribonucleotide as well. The 5′ segment of Primer 2 is labeled as Segment 3 (thick double line) and the 3′ segment as Segment 4 (thin double line). In the presence of Template 1 and Template 2 of a target DNA, each of the primer anneals to its template and is extended by the polymerase (FIG. 9, b). The extension of Primer 1 from Segment 2 produces Amplicon 1 from Template 1. In the meantime, the extension of Primer 2 from Segment 3 produces Amplicon 2 from Template 2 (FIG. 9, b). The ribonucleotides in Primer 1 and Primer 2 are then recognized and cleaved at the 5′ side of ribonucleotides to make Segment 1 in Primer 1 and Segment 3 in Primer 2 extendable. Extension from Segment 1 and Segment 3 produce Amplicon 3 and 4 respectively while displacing the corresponding Amplicon 1 and Amplicon 2 generated by the extension of Segment 2 and Segment 4 (FIG. 9, c). Amplicon 3 and 4 mostly remain as double stranded and are not used for further amplification unless denatured as depicted in FIG. 10. Displaced Amplicon 1 anneals with another Primer 2 while displaced Amplicon 2 anneals with another Primer 1 (FIG. 9, c). Extension of the annealed Primer 2 generates Amplicon 5 off Amplicon 1 and extension of the annealed Primer 1 generates Amplicon 6 off Amplicon 2 (FIG. 9, d). Recognition and cleavage of the extended Primer 2 and Primer 1 by RNase H2 make the Segment 3 in Primer 2 and Segment 1 in Primer 1 extendable, and their extensions by the polymerase produce Amplicon 7 and Amplicon 8 (FIG. 9, e) while displacing Amplicon 5 and Amplicon 6 respectively (FIG. 9 e). The displaced Amplicon 5 then anneals with the Segment 2 of a Primer 1, and Amplicon 6 with the Segment 4 of a Primer 2 (FIG. 9, e; Box1). Extension of the 3′ end of Amplicon 5 makes Segment 1 double stranded, and extension of Segment 2 of Primer 1 off Amplicon 5 produces Amplicon 9 (FIG. 9, f). Similarly, extension of Amplicon 6 makes Segment 3 double stranded, and extension of Segment 4 of Primer 4 off Amplicon 6 produces Amplicon 10 (FIG. 9, f). The ribonucleotides within the double stranded regions are then recognized and cleaved by RNase H2 (not shown), and the Segment 1 in Primer 1 and Segment 3 in Primer 2 are next extended to produce Amplicon 11 and Amplicon 12 respectively (FIG. 9, g) while Amplicon 9 and Amplicon 10 are displaced and annealed to Segment 4 of a Primer 2 and Segment 2 of a Primer 1 respectively (FIG. 9, g; Box2). The structures in Box2 are the same as those in Box1 and regenerate the same structures through Step f. As a result, these structures are continuously cycled to produce increased amount of amplicons at a constant condition. The amplification rate is increased when one or both of the primers are designed to have more segments separated by additional ribonucleotides.

FIG. 10 shows one of the applications of the amplification method depicted in FIG. 9. One of the ribonucleotide containing primers is designed to include a tag. The tagged primer is used to reverse transcribe an RNA target. (FIG. 10). When an RNA polymerase promoter is used as the tag, the initial incorporation of the promoter into double strand DNA and the in vitro transcription (IVT) may be carried out in the same tube at the same temperature. Reverse transcriptase may be additionally provided to amplify the target nucleic acids by Nucleic Acid Based Amplification (NASBA). A pair of ribonucleotide containing primers are designed to amplify the target region (FIG. 10, a). Either DNA or RNA can be used as starting template. An RNA template is shown in the figure. One of the primers (shown as the forward primer) is designed to have two segments separated by a ribonucleotide and each segment matches the target sequence. Another primer (shown as the reverse primer) is designed to one ribonucleotide in the 5′ tag. The 5′ tag consists of an RNA polymerase promoter that RNA polymerase uses to initiate in vitro transcription (IVT). The 3′ end that is connected with the promoter is designed to anneal to the template to initiate reverse transcription (FIG. 10, a). Reverse transcriptase is used to convert the RNA into cDNA and its RNase H activity or natural transient dissociation of the RNA from the cDNA enables the annealing of the forward primer and extension of the primer, either by reverse transcriptase activity or added DNA polymerase (FIG. 10, b). The extension of the forward primer creates the double stranded RNA polymerase promoter (FIG. 10, c). The promoter is then used by RNA polymerase (FIG. 10, d) to transcribe the target sequence into multiple copies of RNA (FIG. 10, e). The presence of ribonucleotides in the primers leads to target amplification at the same temperature as shown in FIG. 9. The newly transcribed RNA shown in FIG. 10, e further serves as template for reverse transcription by the forward primer and the cDNA thus produced are amplified further by the ribonucleotide containing forward primer (FIG. 10, g). Any additionally generated double strand promoter leads to IVT by the RNA polymerase, and the RNA are further reverse transcribed into cDNA and fuels next round of amplification. The cycles of reverse transcription and IVT plus the amplification by the ribonucleotide containing primers synergically speed up the amplification of target nucleic acids drastically. Use of the ribonucleotide containing primers foregoes the need of initial thermocycling and enables the amplification at relatively constant temperatures.

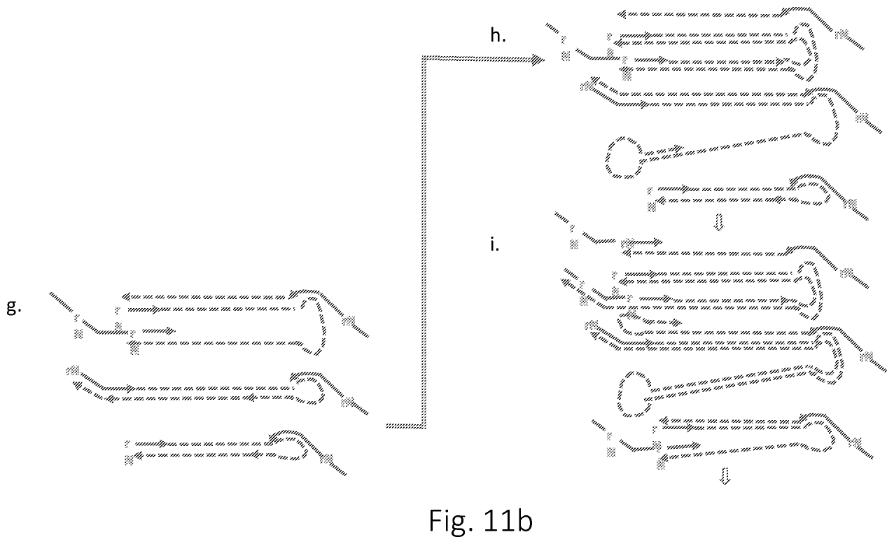

FIG. 11 shows the isothermal amplification of target using loop primers as typically used in LAMP assays. In this example, only two or three primers are needed (FIG. 11a, a). Primer B3 is optional. The forward internal primer (FIP) and the backward internal primer (BIP) are each designed to have a 5′ segment that forms a double strand stem (e.g., looping) once being extended on a targeted template. The two primers generally follow the LAMP inner primer designs with some modifications. In the FIP, a ribonucleotide is incorporated close to the center of the F2 and F1c region. The Tm of each segment within F2 and F1c is high enough to form internal loop structure at the designated reaction temperature. In the BIP, a ribonucleotide is incorporated in the B1c region and each half of B1c segment can form internal loop structure after cleavage of the ribonucleotide. A third optional primer B3 can be used to first displace the extension product of BIP (FIG. 11a, b). Once BIP extension product is dissociated from its template, FIP anneals to and copies the displaced BIP extension product (FIG. 11a, c). RNase H2 cleavage of FIP and extension of F1c 5′ segment and displacement of the first extension product of FIP generates looped structure in FIG. 11a, d, which is further extended to form long looped structure in FIG. 11, e. Cleavage and extension of BIP primer leads to two additional amplicons in FIG. 11, e. Each of those structures is further annealed and extended in ensuing steps (FIG. 11a, f & g; FIG. 11b), leading to amplification of the target.

### Strand Displacement Chain Reaction (SDCR)

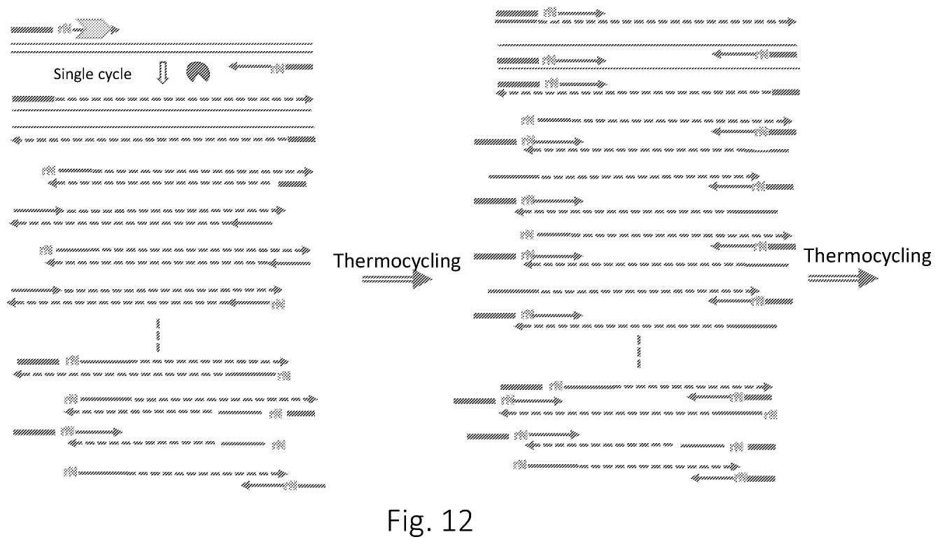

FIG. 12 shows an example application of the process shown in FIG. 10. In this example, thermocycling is used to denature the double stranded amplicons to initiate additional rounds of amplification. When both forward and reverse primer are ribonucleotide containing primers, the target nucleic acids may be amplified by strand displacement chain reaction (SDCR) (FIG. 12). At each circle, the template is denatured first at a temperature to dissociate double stranded DNA, followed by the annealing and extension of the primers. The annealing and extension may be carried out at the same temperature. Each template is replicated more than once as shown in FIG. 10, and the fold of increase at each cycle is dependent on the annealing and extension time. By implementing a denaturation step by thermocycling, all the double stranded amplificons are denatured and serve as template to anneal to the primers. At the annealing and/or extension step, the amplification by the ribonucleotide containing primers continues in a similar efficiency of the first cycle. When the ribonucleotide containing primers are all cleaved and extended at each cycle, the amplification efficiency is doubled as compared to standard PCR. When more than one copy is generated from one template at each cycle, the amplicon copies increase exponentially with a larger than 2 bases.

### Polymerase

The disclosed methods make the use of a polymerase for amplification. In some embodiments, the polymerase is a DNA polymerase that lacks a 5′ to 3′ exonuclease activity but has “strand displacement” activity. The polymerase is used under conditions such that the strand extending from a first primer can be displaced by polymerization of a second primer provided or generated by RNase H2 cleavage. FIG. 2-12 illustrates the embodiments making use of strand displacement polymerases.

Conveniently, the polymerase is capable of displacing the strand complementary to the template strand, a property termed “strand displacement.” Strand displacement results in synthesis of multiple copies of the target sequence per template molecule. Exemplary polymerases with strand displacement activity include M-MuLV reverse transcriptase, phi29 DNA polymerase, DEEP VENT (exo-) DNA polymerase (all available from NEB), SD polymerase (Bioron). If thermocycling is to be carried out (as in PCR), the polymerase is preferably a thermostable polymerase.

Strand displacement can also be facilitated through the use of a strand displacement factor, such as a helicase. Any DNA polymerase that can perform strand displacement in the presence of a strand displacement factor is suitable for use in the disclosed method, even if the DNA polymerase does not perform strand displacement in the absence of such a factor. Strand displacement factors useful in the methods described herein include BMRF1 polymerase accessory subunit (Tsurumi et al., J. Virology 67 (12): 7648-7653 (1993)), adenovirus DNA-binding protein (Zijderveld and van der Vliet, J. Virology 68 (2): 1158-1164 (1994)), herpes simplex viral protein ICP8 (Boehmer and Lehman, J. Virology 67 (2): 711-715 (1993); Skaliter and Lehman, Proc. Natl. Acad. Sci. USA 91 (22): 10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)). Helicase and SSB are available in thermostable forms and therefore suitable for use in PCR.

In some embodiments, it can be advantageous to use a blend of two or more polymerases. For example, an illustrative polymerase blend includes a polymerase that is particularly proficient at initiating extension from a partially double-stranded DNA primer and a polymerase that is particularly proficient at strand displacement synthesis, since combining these properties may provide a net advantage in some embodiments. In some embodiments, a reverse transcriptase is combined with a DNA polymerase. The DNA polymerase and the reverse transcriptase may have strand displacement activity.

In some embodiments, the DNA polymerase for use in the disclosed methods is highly processive. Exemplary DNA polymerases include variants of Taq DNA polymerase that lack 5′ to 3′ exonuclease activity, e.g., the Stoffel fragment of Taq DNA polymerase (ABI), SD polymerase (Bioron), mutant Taq lacking 5′ to 3′ exonuclease activity described in U.S. Pat. No. 5,474,920, Bca polymerase (Takara), Pfx50 polymerase (Invitrogen), Tfu DNA polymerase (Qbiogene). If thermocycling is to be carried out (as in PCR), the DNA polymerase is preferably a thermostable DNA polymerase.

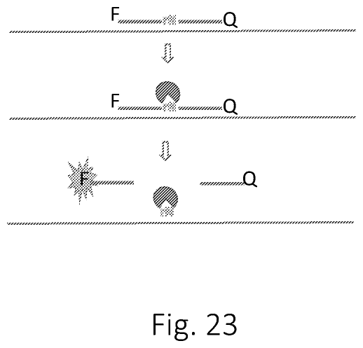

In a different aspect, where it is desirable to use a Taqman-style probe to carry out real-time PCR, a polymerase blend can include a polymerase that has 5′ to 3′ exonuclease activity, provided the primer structure is designed so that it is not susceptible to “flap” endonuclease activity. In some embodiments, the probe has one or more ribonucleotides in the sequence (see an example shown in FIG. 23). The probe may be designed to hybridize with the amplicons and generate greater signal upon cleavage by RNase H2. In some embodiments, more than one probe may be used to hybridize to different sequences of the amplicon. The probes may be labeled with different dyes.

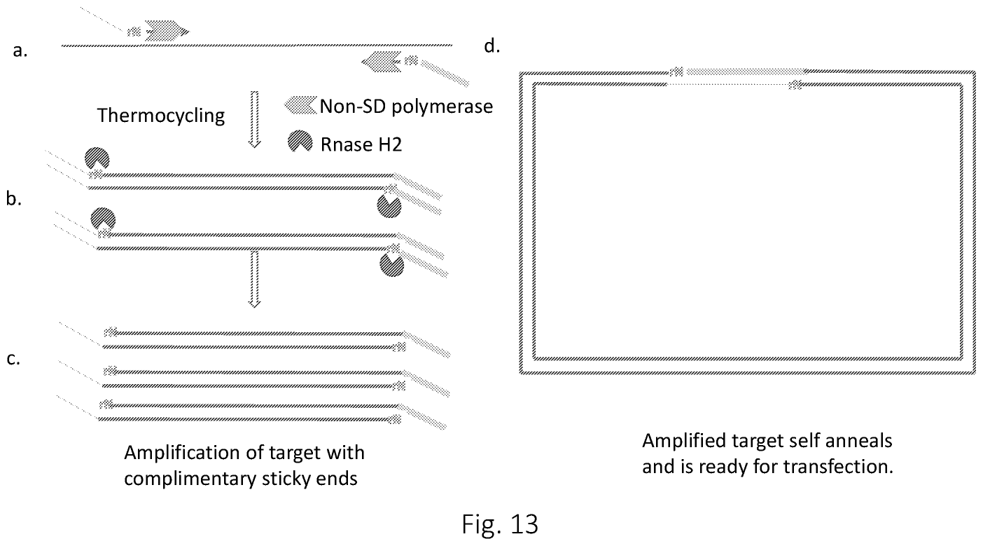

In some embodiments, the ribonucleotide containing primers are used with DNA polymerases that lack strand displacement activity (FIGS. 13, 14, 16, 16, 17, 18, 19). The ribonucleotide containing primers may be used in a PCR in the presence of thermostable RNase H2. The 5′ end segment is cleaved once the ribonucleotide containing primer is incorporated into a double stranded amplicon. Unlike polymerases with strand displacement activities, non-strand displacement polymerase cannot extend the cleaved 5′ end segment. As a result, the cleaved 5′ end segments can then dissociate from the amplicons and serve as primers. Once the cleaved 5′ end segment is dissociated, the amplicon is left with a single stranded 3′ end.

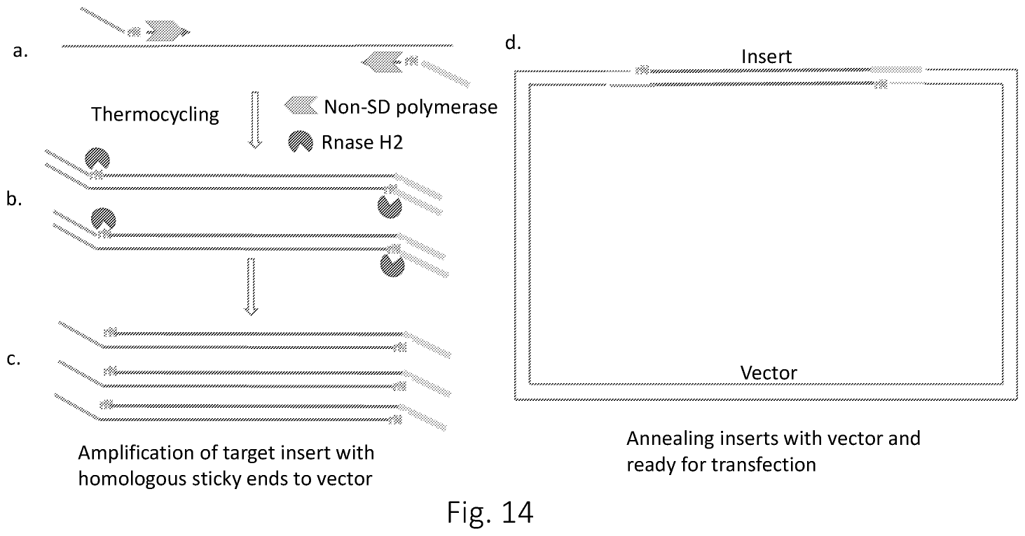

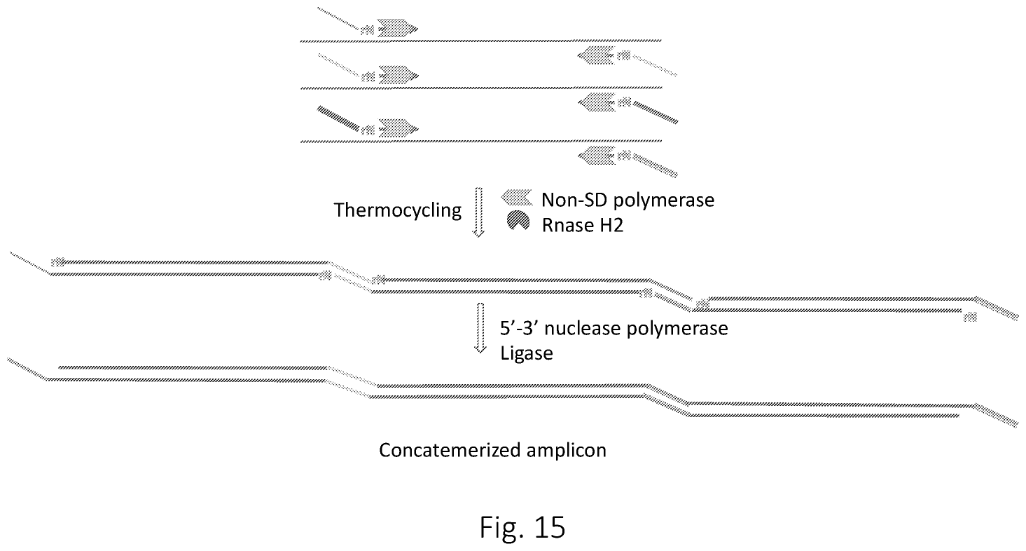

In some embodiments, the forward primer and the reverse primer are designed to each have a tag (FIG. 13, 14) 5′ to the respective locus-specific 3′ end. The locus-specific segments have appropriate Tm to anneal to the target at the designated conditions. The tags may be joined to a locus-specific sequence through a ribonucleotide at the 3′ end (FIG. 13a). They may be designed to hybridize with each other so that each amplicon has the two ends hybridizing with each other to form a circle. During PCR amplification through thermocycling, the ribonucleotide is recognized and cleaved at its 5′ side by RNase H2 (FIG. 13b). The 5′ end segment dissociates from the PCR amplicons, leaving both of the 3′ ends single stranded (FIG. 13c). The amplificons can anneal through intramolecular annealing of the two 3′ ends and thus forms a circle (FIG. 13d). The amplicon circles can be further transfected into cells directly or after ligation. In a preferred embodiment, the circle can be processed and ligated to seal the nicks. In some embodiments, the tags are designed to hybridize with a provided cloning vector with single stranded “sticky” ends (FIG. 14). The 5′ ends of the two PCR primers are designed to form complementary sequences with the vector (FIG. 14, a). The target is amplified with PCR with a non-strand displacement polymerase in the presence RNase H2. The amplicons (FIG. 14, b) will have complementary “sticky” ends to the vector upon cleavage by RNase H2 (FIG. 14, c). The amplicons may be mixed with the vectors for transformation or transfection of recipient cells (FIG. 14, d). In a preferred embodiment, the annealed amplicon and vector can be processed and ligated to seal the nicks. In some other embodiments, multiple amplicons are concatemerized by using ribonucleotide containing primers (FIG. 15). Ribonucleotide containing primer pairs are designed to amplify more than one target sequences. Each locus-specific 3′ segment of the primer is designed to connect to a 5′ end segment (FIG. 15, a). The 5′ end segment of the primers may be designed to hybridize with one of the other amplicons so that once incorporated into the amplicons and digested by RNase H2, the amplicons can join each other in pre-determined order (FIG. 15, b). Multiplex PCR can be used to amplify more than one target sequences. When the 5′ segments are cleaved by RNase H2, they can anneal among themselves to form predesigned sequences.

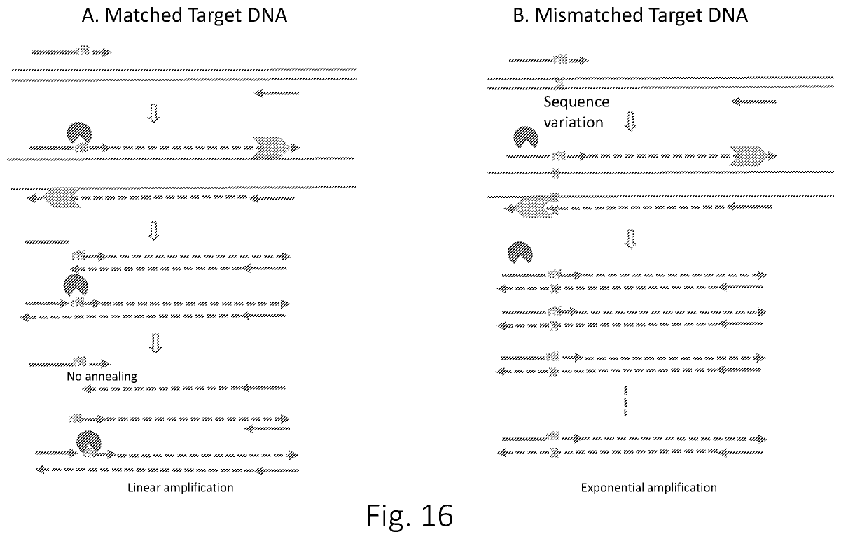

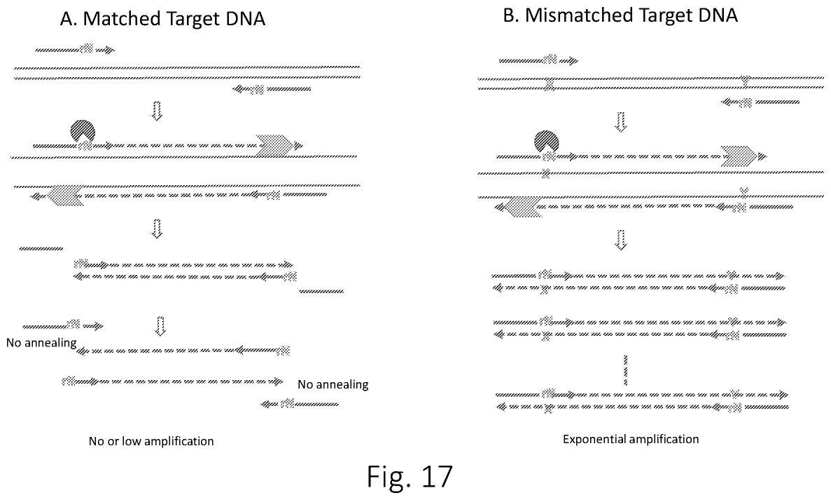

In some embodiments, the ribonucleotide containing primer is used to differentiate or enrich a variation in the target nucleic acid (FIG. 16). One ribonucleotide containing primer is used in a pair of primers to amplify the targeted sequence by PCR. The ribonucleotide is designed to overlap or in the near vicinity of the targeted sequence variation (indicated by a X in the figure). The segment to the 3′ side of the ribonucleotide is designed to be short so its Tm is lower than the annealing temperature. When the primer is perfectly matched with the target, the primer is cleaved by a RNase H2 (FIG. 16, A). The amplicons generated from the cleaved sequence will not stably anneal with the primer in the ensuing circles, resulting in close-to-linear amplification of the perfectly matched template. On the contrary, when the primer is mismatched to the target due to the presence of sequence variations, the cleavage is hindered or diminished. The amplification of the mismatched template proceeds as in a standard exponential amplification (FIG. 16, b). In some further embodiments, the genotypes of the variation can be determined by the pair of ribonucleotide-containing primers. Efficient amplification indicates the presence of a sequence variation at the ribonucleotide site. In another embodiment, variants that differs from the primer sequences are preferentially amplified and enriched. In some further embodiments, each of the forward and reverse primer targets one of the variations in the target nucleic acid (FIG. 17). The method is useful to determine the haplotype of the target sequence. Efficient amplification is observed when two variations are within the same strand. A haplotype can be deducted from the amplification of the target and the identities of the ribonucleotides in the primers.

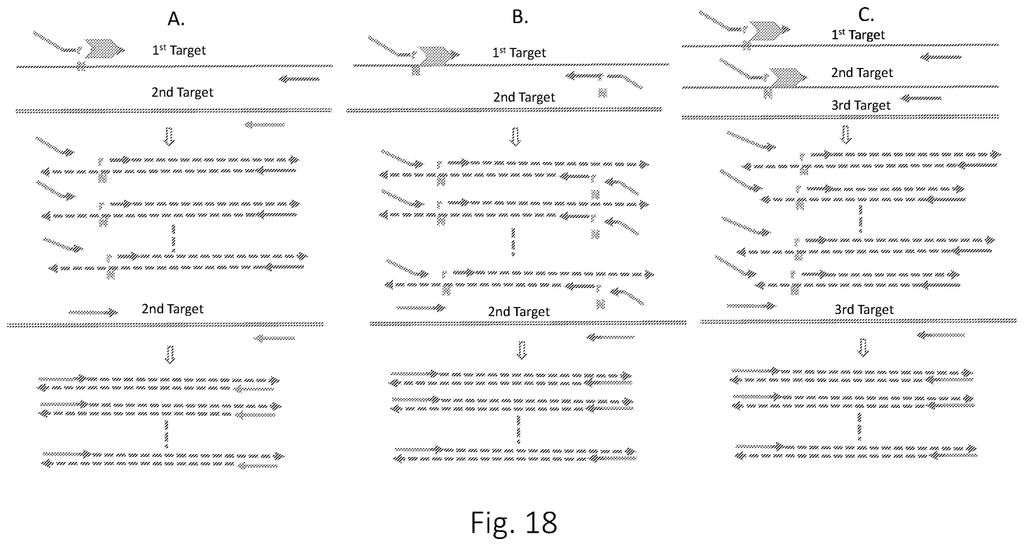

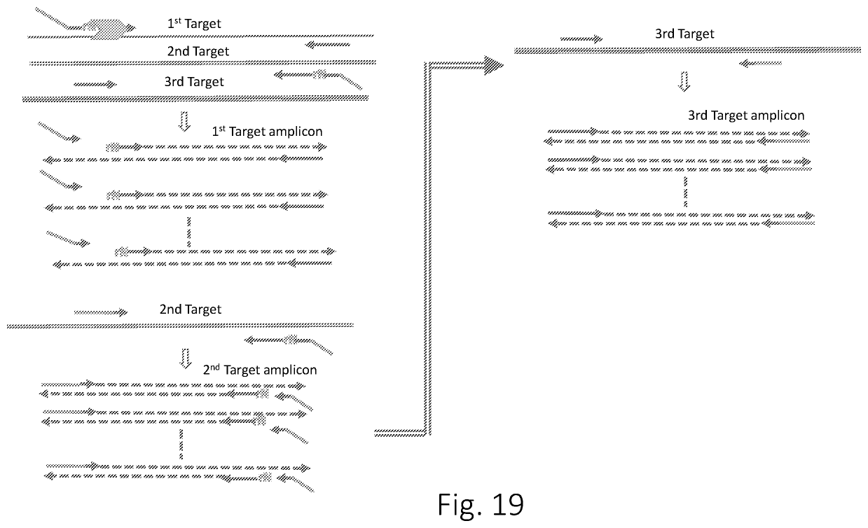

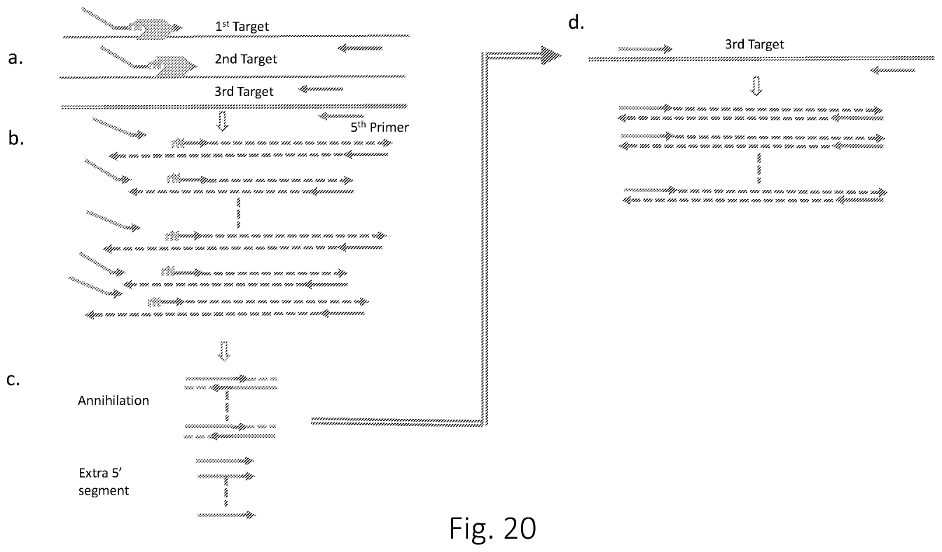

In some embodiments, the ribonucleotide containing primer can be used to initiate cascade of reactions (see FIGS. 18 and 19). These methods are useful multiplex PCR alternatives for the detection of multiple targets. In these designs, ribonucleotide cleavage at the 5′ side by RNase H2 in amplicons releases the 5′ end segment as new primers for further reactions. In one example (FIG. 18A), the cleaved 5′ segments of one of the primer pair for a first target forms a primer pair with a third primer to amplify a second template. The amplification of the first template supplies the needed primer to amplify the second template. The amplification of the second target is dependent of the presence and amplification of the first target. As a result, amplification and detection of the second template indicates the presence of the first template and the second template. FIG. 18B shows a variation of FIG. 18A. In this example, two ribonucleotide containing primers are designed to amplify the first target. The 5′ segments from both primers targeting the first template are used to amplify a second target. Amplification of the first target supplies the primers to amplify the second target. In yet another embodiment (FIG. 18C), the 5′ segments from two primers from two separate primer pairs targeting the first and second templates are designed to amplify the third target. The amplification and detection of the third target is dependent upon the presence and amplification of the first two templates. In another example as shown in FIG. 19, the 1^{st} target is amplified with a first pair of primers. One primer is designed as ribonucleotide containing primer. The 5′ end segment of the first ribonucleotide containing primer forms a primer pair with a third primer to amplify a second target. The third primer is designed as a ribonucleotide containing primer with its 5′ end segment designed to form a primer pair with a fourth primer to amplify a third target. The cascade of amplification and releasing 5′ segments as new primers lead to the amplification of the third target. As a result, only when the first target and the second target are present at appropriate amount is the third target amplified. The amplification and detection of the third target indicates the presence of the first and second target. In another embodiment (FIG. 20), the amplification cascade is used to detect target proportion difference. Two targets that potentially have unequal proportion are amplified by two primer pairs (FIG. 20, a). One primer of each pair is designed as a ribonucleotide primer. The 3′ half of the 5′ end segments are designed to be complementary so they can anneal to each other and “annihilate” each other at 1:1 ratio. When the proportions of the first two templates are different, different amount of the 5′ segments are produced by the amplification of the two targets. The extra amount of one of the 5′ end segment serves as one of the primers to amplify a third target with a fifth primer. Amplification and detection of the third template indicates the unequal proportion of the first and second template. This method is useful for copy number variation and trisomy detection.

Illustrative polymerase concentrations range from about 0.1 to 200 units per reaction. In various embodiments, the polymerase concentration can be at least: 0.1, 0.5, 1, 5, 10, 20, 30, 40, 50, 60, 70, 80, 90, 100, 110, 120, 130, 140, 150, 160, 170, 180, 190, or 200 or more units per reaction. In some embodiments, the polymerase concentration falls within a range bounded by any of these values, e.g., 0.1-1, 1-10, 10-200, 10-150, 10-100, 10-50, 20-150, 20-100, 20-50, 50-200, 50-150, 50-100, 100-200, 100-150, etc. units per reaction. When polymerase blends are used, the total, combined polymerase concentration can be any of these values or fall within any of these ranges.

### Amplification

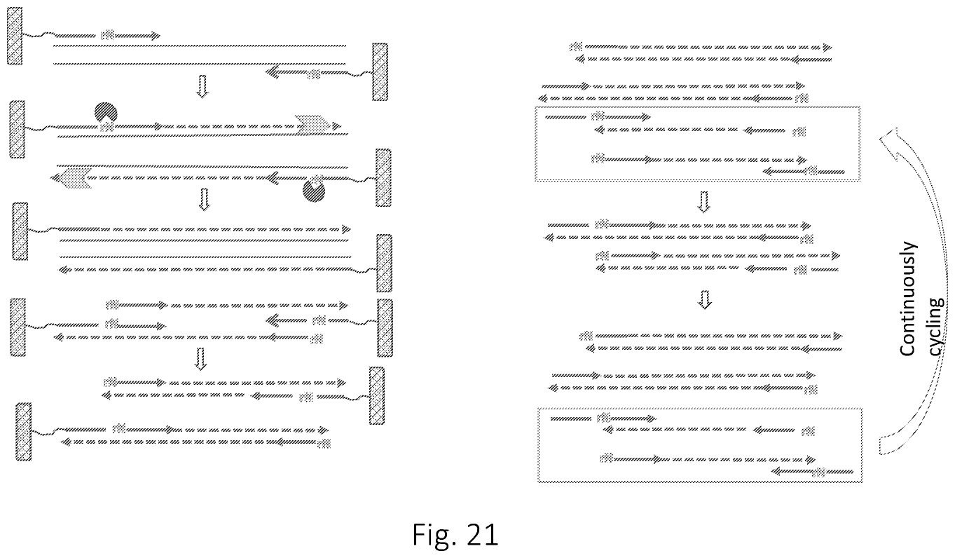

The primer sets described above are contacted with sample nucleic acids under conditions wherein the primers anneal to their template strands, if present. The desired nucleic acid amplification method is carried out using a DNA polymerase lacking 5′-3′ exonuclease activity under the reaction conditions employed. In some embodiments, strand displacement polymerase is advantageous. In other embodiments, non-strand displacement polymerase is advantageous. Reaction mixtures with appropriate components and buffer conditions are provided to support polymerase and RNase H activities. The primer sets can be conveniently added to the amplification mixture in the form of separate oligonucleotides. For example, the two-primer set can consist of one or two ribonucleotide containing primers. The reaction may be carried out in solution or with one or two primers fixed on a matrix (FIG. 21, 22).

In some embodiment, the amplification step is performed isothermally. Isothermal amplification may be performed as strand-displacement amplification, NEAR, LAMP, RT LAMP, NABSA, RPA (recombinase polymerase amplification). RNase H2 and ribonucleotide containing primers can be used in place of all-deoxynucleotide primers in their specific buffers.

In some embodiments, the amplification step is performed using PCR. For running real-time PCR reactions, reaction mixtures generally contain an appropriate buffer, a source of magnesium ions (Mg²⁺) in the range of about 1 to about 10 mM, e.g., in the range of about 2 to about 8 mM, nucleotides, and optionally, detergents, and stabilizers. An example of one suitable buffer is TRIS buffer at a concentration of about 5 mM to about 85 mM, with a concentration of 10 mM to 50 mM preferred. In one embodiment, the TRIS buffer concentration is 80 mM in the reaction mix double-strength (2×) form. The reaction mix can have a pH range of from about 7.5 to about 9.0, with a pH range of about 8.0 to about 8.5 as typical. Concentration of nucleotides can be in the range of about 25 μM to about 1000 μM, typically in the range of about 100 μM to about 800 μM. Examples of dNTP concentrations are 100, 200, 300, 400, 500, 600, 700, and 800 μM. Detergents such as Tween 20, Triton X 100, and Nonidet P40 may also be included in the reaction mixture. Stabilizing agents such as dithiothreitol (DTT, Cleland's reagent) or mercaptoethanol may also be included. In addition, master mixes may optionally contain dUTP as well as uracil DNA glycosylase (uracil-N-glycosylase, UNG). A master mix is commercially available from Applied Biosystems, South San Francisco, Calif., (TaqMan Universal Master Mix, cat. nos. 4304437, 4318157, and 4326708).

### Labeling Strategies

Any suitable labeling strategy can be employed in the methods described herein. Where the reaction is analyzed for presence of a single amplification product, a universal detection probe can be employed in the amplification mixture. In particular embodiments, real-time PCR detection can be carried out using a universal qPCR probe. Suitable universal qPCR probes include double-stranded DNA-binding dyes, such as SYBR Green, Pico Green (Molecular Probes, Inc., Eugene, Org.), Eva Green (Biotium), ethidium bromide, and the like (see Zhu et al., 1994, Anal. Chem. 66:1941-48). In some embodiments, colorimetric dyes may be used to detect amplification. pH changes may be used to indicate amplification. A pH indicator may be used to show color changes when amplification causes pH shift. A colorimetric dye that is sensitive to free Mg²⁺ concentration changes may so be used. In some embodiments, luminescence may be used to detect the amplification.

In some embodiments, one or more target-specific qPCR probes (i.e., specific for a target nucleotide sequence to be detected) is employed in the amplification mixtures to detect amplification products. By judicious choice of labels, analyses can be conducted in which the different labels are excited and/or detected at different wavelengths in a single reaction (“multiplex detection”). See, e.g., Fluorescence Spectroscopy (Pesce et al., Eds.) Marcel Dekker, New York, (1971); White et al., Fluorescence Analysis: A Practical Approach, Marcel Dekker, New York, (1970); Berlman, Handbook of Fluorescence Spectra of Aromatic Molecules, 2nd ed., Academic Press, New York, (1971); Griffiths, Colour and Constitution of Organic Molecules, Academic Press, New York, (1976); Indicators (Bishop, Ed.). Pergamon Press, Oxford, 19723; and Haugland, Handbook of Fluorescent Probes and Research Chemicals, Molecular Probes, Eugene (1992); and Linck et al. (2017) “A multiplex TaqMan qPCR assay for sensitive and rapid detection of phytoplasmas infecting Rubus species,” PLOS One 12 (5).

In some embodiments, it may be convenient to include labels on one or more of the probes employed in in amplification mixture.

### Exemplary Automation and Systems

In some embodiments, a target nucleic acid is detected using an automated sample handling and/or analysis platform. In some embodiments, commercially available automated analysis platforms are utilized. For example, in some embodiments, real-time qPCR systems are utilized. However, the present invention is not limited to a particular detection method or analysis platform. One of skill in the art recognizes that any number of platforms and methods may be utilized.

In some embodiments, a target nucleic acid is detected using a lateral flow device. A sample contains a target nucleic acid is added to the cartridge, the sample is contacted with lysis buffer and released nucleic acid is shifted along the lateral flow cartridge. The nucleic acid may then contact with reaction mixes deposited in the cartridge to initiate amplification. The amplified products may be then detected by fluorescence, luminescence or color changes.

Components of the cartridge include, but are not limited to, spots or lines containing lysis reagents, filters, and capture technologies useful to extract, purify, and amplify target nucleic acids. An optical window enables detection.

### Kits

Also contemplated is a kit for carrying out the methods described herein. Such kits include one or more reagents useful for practicing any of these methods. A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

In some embodiments, a kit can include one or more ribo-primers as disclosed herein, a polymerase described herein, and a RNase as described herein.

Kits preferably include instructions for carrying out one or more of the amplification methods described herein. Instructions included in kits can be affixed to packaging material or can be included as a package insert. While the instructions are typically written or printed materials, they are not limited to such. Any medium capable of storing such instructions and communicating them to an end user can be employed. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

### Example 1. R-Primer Amplified Target Similarly with Standard Primer but Shifted Ct Higher and Generated Shorter Amplicons in the Presence of RNase H2

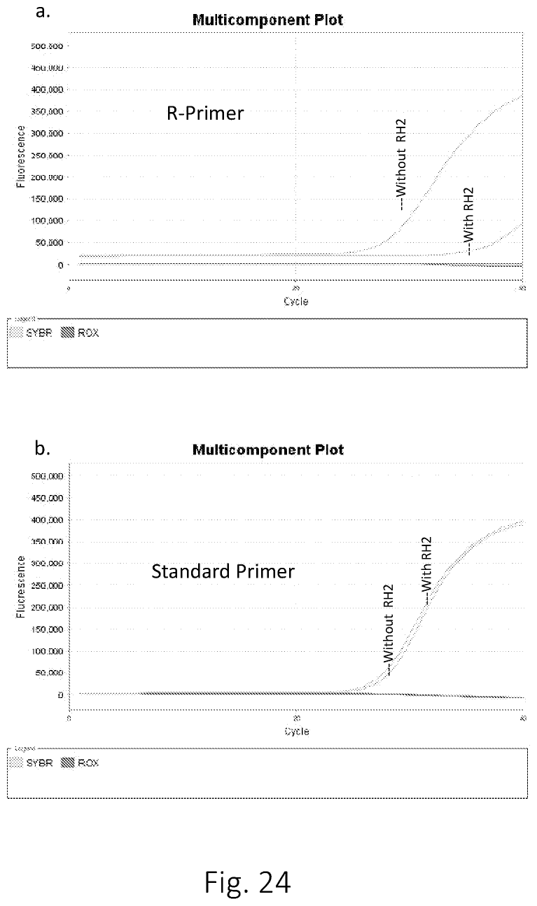

Covid-19 N gene plasmid (IDT, 2019-nCoV_N_Positive Control, Catalog #10006625) was amplified with primers that targeted the N gene sequence within the plasmid. Standard primers (Forward primer: ACT GAG GGA GCC TTG AAT ACA (SEQ ID NO:1); Reverse primer: TGC AGC ATT GTT AGC AGG AT (SEQ ID NO:2)) was compared to R-primer (same standard primer; R-primer: ACT GAG GGA GCrC TTG AAT A+CA, +: LNA (SEQ ID NO:3)) in the amplification of the target sequence. In each 10 ul reaction, 500 nM of forward primer and 500 nM of reverse primer were used to amplify 2000 copies of N gene plasmid in 1× Q5 Hot Start High-Fidelity Master Mix (NEB, Catalog #M0494) in the presence of 1×SYBR and 30 nM ROX. For reactions with RNase H2, 2.6 mU/ul of RNase H2 (IDT, catalog #Nov. 3, 2002-02) was added in the reaction. The reactions were thermos cycled on a StepOnePlus Real Time PCR System (Thermo Fisher Scientific, Catalog #4376600) and the amplification was monitored at SYBR fluorescence channel. The thermocycles were 95° C./30 sec, 40 cycles of 95° C./15 sec and 65° C./1 min. The amplification curves were plotted as SYBR fluorescence intensity vs cycle number in Multicomponent Plot view, see FIG. 24.

As shown in FIG. 24, standard all-DNA primers amplified similarly in the absence or presence of RNase H2 (plot on the right). R-primer functioned similarly with the standard primers in the absence of RNase H2 but had much reduced amplification in the presence of RNase H2 (plot on the left).

### Example 2. R-Primer Generated Shorter Amplicons in the Presence of RNase H2

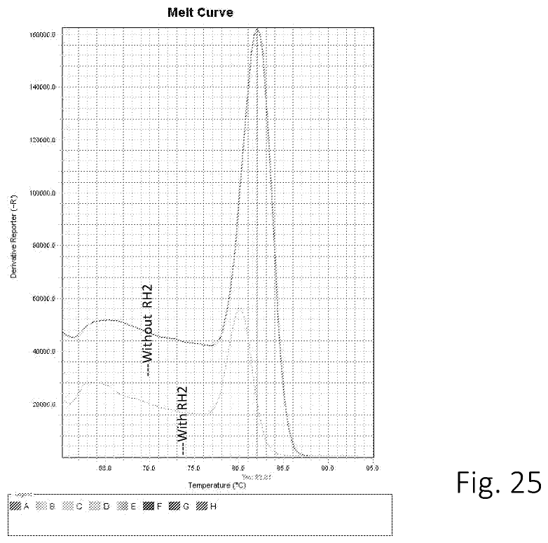

Melt curves of the amplification products with R-primer in Example 1 were collected on the StepOnePlus Real Time PCR System after the thermocycling steps. See FIG. 25. Amplicons generated in the presence of RNase H2 has lower Tm than those generated in the absence of RNase H2, indicating the amplicons generated in the presence of RNase H2 were shorter and the single ribonucleotide in the R-primer incorporated into the double stranded amplicon was cleaved by RNase H2.

### Example 3. Specific Detection of EGFR Exon21 Cancer Mutation with R-Primer and Non-SD Polymerase

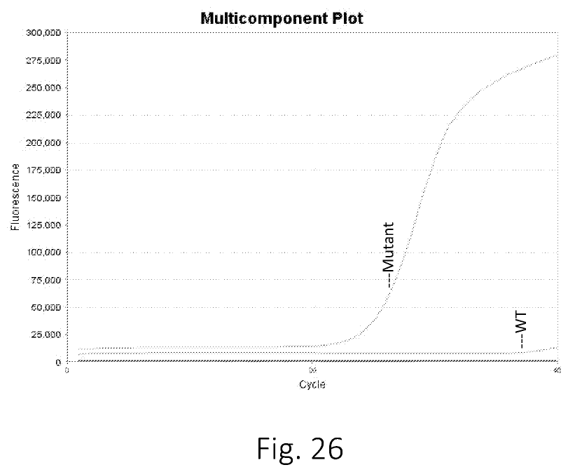

Human genomic DNA was amplified with a pair of primers, one of which was a F-primer. Primers were designed to amplify the sequences of human EGFR gene exon 21. The forward R-primer (CAA GAT CAC AGA TTT TGG GCrU GGC (SEQ ID NO:4)) was designed to have the ribonucleotide base located at the base that is mutated from wildtype T to cancerous G. The R-primer was used with reverse primer (TTT CTC TTC CGC ACC CAG (SEQ ID NO: 5)) in 1×Q5 Hot Start High Fidelity Master Mix to amplify EFGR exon 21 sequence. The reaction consisted of 500 nM each of the forward R-primer and reverse primer, 2 ng human genomic DNA in 1× master mix and 1×SYBR. For the reaction with RNase H2, 2.6 mU/ul of RNase H2 was added to each reaction. The reactions were thermo cycled at the conditions of 95° C./2 min, 40 cycles of 95° C./15 sec, 60° C./30 sec and 72° C./1 min on a StepOnePlus Real Time PCR System (Thermo Fisher Scientific). As shown in FIG. 26, in the presence of RNase H2, the amplification of the wild-type sequence was greatly reduced while the mutant was effectively amplified.

### Example 4. Signal Growth was Increased by r-BIP and RNase H2 in LAMP

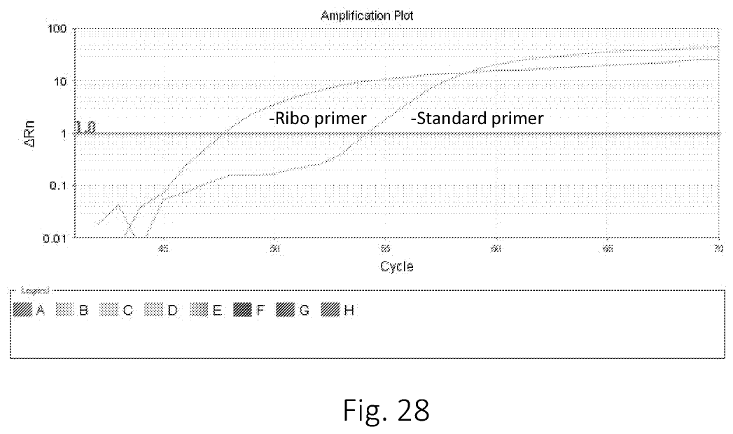

LAMP assay was designed to amplify SARS-CoV-2 N gene. One ribonucleotide was incorporated into each of two of the primers, B3 and BIP (B3: TGC AGC ATT GrUT AGC AGG+AT (SEQ ID NO:6); BIP: AGA CGG CAT rCAT ATG GGT TGC ACG GGT GCC AAT GTG AT+C T (SEQ ID NO:7)), while the other four, F3, FIP, LF and LB, were standard primers (F3: TGG CTA CTA CCG AAG AGC T (SEQ ID NO:8); FIP: TCT GGC CCA GTT CCT AGG TAG TCC AGA CGA ATT CGT GGT GG (SEQ ID NO:9); LF: GGA CTG AGA TCT TTC ATT TTA CCG T (SEQ ID NO:10); LB: ACT GAG GGA GCC TTG AAT ACA (SEQ ID NO:1)). In the presence of RNase H2, the R-primer reduced the time of detection (FIG. 28).

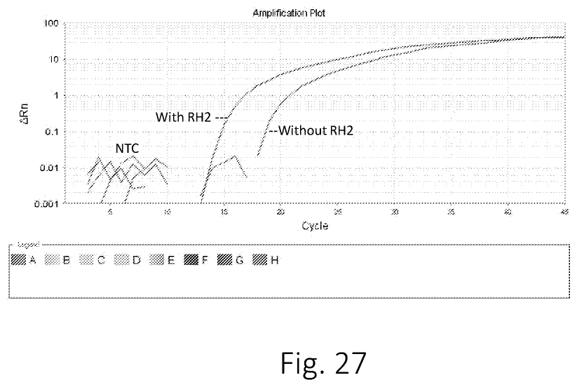

Primer concentrations in the final reaction were 200 nM for F3 and B3, 1.6 μM FIP and BIP, and 400 nM LF and LB. The primers were pre-annealed with 2000 copies of Covid-19 N gene plasmid (IDT, 2019-nCoV_N_Positive Control, Catalog #10006625) with 0.5×SYBR in a volume of 8.31 ul by heating up to 90° C. and cooling to 20° C. with a StepOnePlus Real Timer PCR System. The primers and plasmid mixtures in each reaction well on a 96-well plate were then added WarmStart LAMP 2× Master Mix (NEB, E1700) to 1× and 10 mU/ul RNase H2 (IDT, catalog #Nov. 3, 2002-02) dilute to 0.26 mU/ul or RNase H2 dilution buffer were incubated at 65° C. on a StepOnePlus Real Timer PCR System and fluorescence of SYBR was collected at each cycle for 75 cycles (cycle/minute). Fluorescence was plotted against cycle numbers in the example (FIG. 27). Use of R-primers accelerated the detection of the target.

### Example 5. R-Primers Speed Up LAMP Amplification

LAMP assay was designed to amplify SARS-CoV-2 N gene with 6 primers. The standard primer reaction used all-DNA oligonucleotides: F3: TGG CTA CTA CCG AAG AGC T (SEQ ID NO: 8); B3: TGC AGC ATT GTT AGC AGG AT (SEQ ID NO:2); FIP: TCT GGC CCA GTT CCT AGG TAG TCC AGA CGA ATT CGT GGT GG (SEQ ID NO:9); BIP: AGA CGG CAT CAT ATG GGT TGC ACG GGT GCC AAT GTG ATC T (SEQ ID NO:11); LF: GGA CTG AGA TCT TTC ATT TTA CCG T (SEQ ID NO:10); LB: ACT GAG GGA GCC TTG AAT ACA (SEQ ID NO:1). For the reaction with ribo-primers, one ribonucleotide was incorporated into each of three of the primers, B3, BIP and LB (B3: TGC AGC ATT GrUT AGC AGG+AT (SEQ ID NO:6); BIP: AGA CGG CAT rCAT ATG GGT TGC ACG GGT GCC AAT GTG AT+C T (SEQ ID NO:7); LB: ACT GAG GGA GCrC TTG AAT A+CA (SEQ ID NO:3)), while the other three, F3, FIP and LF, were standard primers (F3: TGG CTA CTA CCG AAG AGC T (SEQ ID NO:8); FIP: TCT GGC CCA GTT CCT AGG TAG TCC AGA CGA ATT CGT GGT GG (SEQ ID NO:9); LF: GGA CTG AGA TCT TTC ATT TTA CCG T (SEQ ID NO:10)). Primer concentrations in the final reaction were 400 nM for F3 and B3, 1.6 μM FIP and BIP, and 400 nM LF and LB. The primers were pre-annealed with 2000 copies of Covid-19 N gene plasmid (IDT, 2019-nCoV_N_Positive Control, Catalog #10006625) with 1×SYBR in a volume of 5.85 ul by heating up to 95° C. and cooling to 20° C. on a StepOnePlus Real Timer PCR System. The primers and plasmid mixtures in reaction wells of a 96-well plate were then added WarmStart Colorimetric LAMP 2× Master Mix (NEB, M1800) to 1×, 10 mU/ul RNase H2 (IDT, catalog #Nov. 3, 2002-02; NEB, catalog #M0288) dilutes to 0.26 mU/reaction and 3.2 U/ul Bsu DNA Polymerase, Large Fragment (NEB, M0330) to 2 U/reaction (total volume 9.15 ul) were incubated at 37° C. for 30 cycles of 37° C./15 sec and 37° C./1 min, 40 cycles of 65° C./15 sec and 65° C./1 min on a StepOnePlus Real Timer PCR System and fluorescence of SYBR was collected at each cycle. Fluorescence was plotted against cycle numbers in the example (FIG. 28). In the presence of RNase H2, the R-primer reduced the time of detection.

### Example 6. SDCR

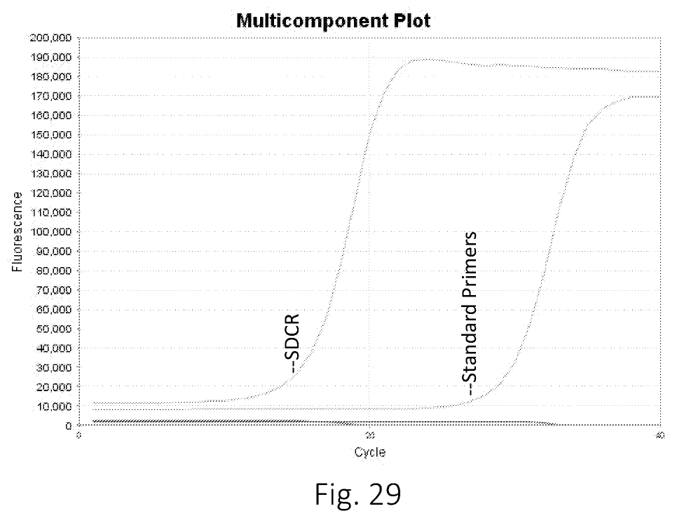

R-primers were used to amplify the N gene cloned in Covid-19 N gene plasmid (IDT, 2019-nCoV_N_Positive Control, Catalog #10006625). Each of the forward primer and reverse primer contained a ribonucleotide in the sequences (Forward r-primer: ACC AAT AGC AGT CCA GAT GAC rCAA ATT GGC TAC TAC CGA AGA GCT (SEQ ID NO:12); Reverse r-primer: GTT CCT TGA GGA AGT TGT AGC ArCG ATT GCA GCA TTG TTA GCA GGA T (SEQ ID NO:13)). The amplification was compared to corresponding standard primers (Forward primer: TGG CTA CTA CCG AAG AGC T (SEQ ID NO:8); Reverse primer: TGC AGC ATT GTT AGC AGG AT (SEQ ID NO:2)). 500 nM of each of the r-primers or standard primers were used to amplify 2000 copies of N gene plasmid in a 10 ul reaction with a strand displacement DNA polymerase, SD Polymerase (Boca Scientific, Catalog #1089100) at 0.3 U/ul in 1×SD DNA polymerase reaction buffer supplemented with 0.375 mM dNTPs, 3.5 mM MgCl2, 1×SYBR and 2.6 mU/ul RNase H2 (IDT, catalog #Nov. 3, 2002-02). The reactions were thermocycled with the protocols of 93 C/3 min, 40 cycle of 60 C/1 min and 65 C/1 min on a StepOnePlus Real Time PCR System (Thermo Fisher Scientific, Catalog #4376600) and the amplification was monitored at SYBR fluorescence channel (FIG. 29). R-primers in combination of a strand displacement DNA polymerase amplified the target with much lower Ct than standard primers in the presence of RNase H2.

### Example 7. Detection of Amplification with Ribo-Probe

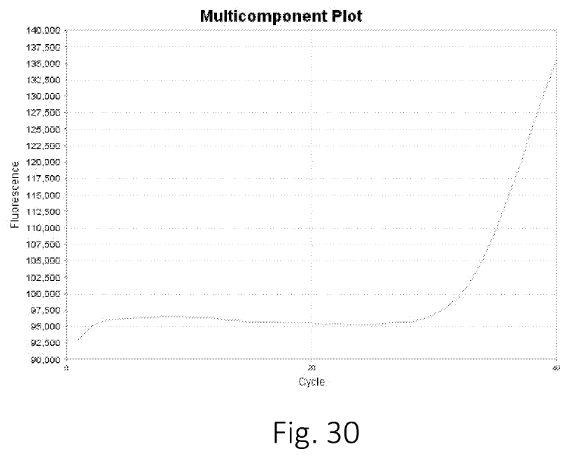

Real time amplification of DNA was detected by probes that consisted of ribonucleotides without the need of 5′-nuclease activities. In a 10 ul reaction, 800 copies of SARS-CoV-2 N gene in Covid-19 N gene plasmid (IDT, 2019-nCoV_N_Positive Control, Catalog #10006625) was amplified with a pair of primers that targeted the N gene (Fw primer: TCT GGC CCA GTT CCT AGG TAG TCC AGA CGA ATT CGT GGT GG (SEQ ID NO:9); Rv primer: AGA CGG CAT CAT ATG GGT TGC ACG GGT GCC AAT GTG ATC T (SEQ ID NO:11)) at 400 nM each in 1×Q5 Hot Start High-Fidelity Master Mix (NEB, Catalog #M0494). Real time amplification was monitored by 400 nM probe (/56-FAM/AGC TGG ACT TCrC rCTA TGG TGC TAA CAA/3BHQ_1/, IDT (SEQ ID NO:14)) in the presence of 5.2 mU/ul RNase H2 (IDT, catalog #Nov. 3, 2002-02) on a StepOnePlus Real Time PCR System. The thermocycle condition was 92° C./2 min, 40 cycles of 95° C./15 sec, 60° C./15 sec and 70° C./1 min. Amplification of the target DNA was detected by the ribo-probe DNA in the presence of RNase H2 using a polymerase that lacks 5′-nuclease activity (FIG. 30).

### Example 8. Detection of SARS-CoV-2 Genes by Real Time SDCR

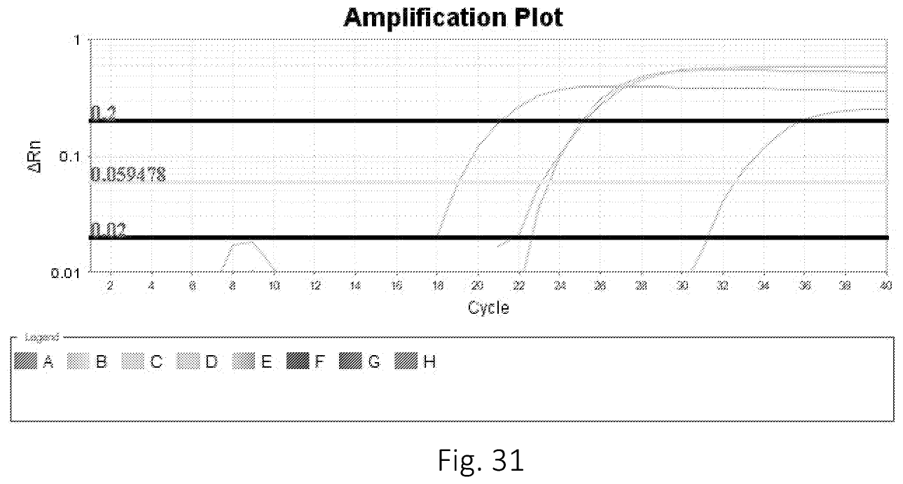

SDCR was used to amplify and detect SARS-CoV-2 E gene and compared to standard primers. Forward primer and reverse primer were selected to target the E gene. Standard primers (Fw primer: GCT TTC GTG GTA TTC TTG CTA GTT (SEQ ID NO:15); Rv primer: GTT AAC AAT ATT GCA GCA GTA CGC A (SEQ ID NO:16)) contained all DNA bases while ribo-primers had the same 3′ half and a 5′ half of similar Tm separated by a ribonucleotide (R-Fw primer: CGT TAA TAG TTA ATA GCG TAC TTC TTT TTC TTIG CTT TCG TGG TAT TCT TGC TAG TT (SEQ ID NO:17); R-Rv primer: CGT AAA AAG AAG GTT TTA CAA GAC TCA CrGT TAA CAA TAT TGC AGC AGT ACG CA (SEQ ID NO:18)). A ribo-probe was selected from the internal sequence between the forward and reverse primers and consisted of a ribonucleotide and labeled with a 5′ fluorophore and 3′ quencher (R-Probe:/5HEX/ACA CTA GCC ATC rCTT ACT GCG CTT CG/3BHQ_1/(SEQ ID NO:19)). To compare SDCR to standard PCR, either each of the forward and reverse was switched with the r-ribo primer or both with ribo primers. The same probe was used for all the reactions. In each 10 ul reaction, 2000 copies of E gene plasmid (IDT, 2019-nCoV_E Positive Control, catalog #10006896) were amplified with 500 nM primers, 250 nM probe, 3 U SD polymerase (Boca Scientific, catalog #108910) and 2.7 mU RNase H2 (IDT, catalog #Nov. 3, 2002-02) in 60 mM Tris-HCl buffer (pH 8.5) supplemented with 625 nM ROX (Lumiprobe, ROX reference dye for qPCR, catalog #31110), 45 mM KCl, 4 mM MgCl2, 0.01% Triton X-100, 100 ng/ul BSA and 0.4 mM dNTPs. The thermocyling conditions were 92° C./2 min, 40 cycles of 60° C./1 min. The amplification was carried out on a StepOnePlus Real Time PCR System (Thermo Fisher Scientific) and monitored by fluorescence increase at VIC channel. Logarithm of ROX normalized HEX fluorescence intensities were plotted with cycle number. As shown in FIG. 31, R-primers reduced the time needed for the detection of the target. From right to left, the amplification curve of A. standard primers, B. r-Fw+standard Rv, C. standard Fw+r-Rv primer, and D. r-Fw+r-Rv primers were shown.

### Example 9. SDCR Amplified Target More than Doubling Per Cycle

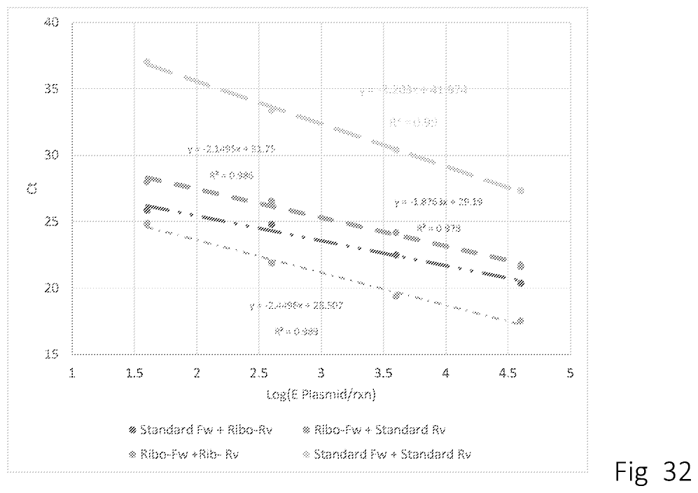

SDCR assays for SARS-CoV-2 E gene were compared to standard primers (FIG. 32). Primer designs and conditions were the same as in Example 8 except the E gene plasmid template amount was titrated at 40,000, 4,000, 400 and 40 copies per reaction. Ct values ware plotted against logarithms of input copy number of template. PCR efficiencies were calculated as 10\hat{ }(−/k), where k was the slope of the linear fit of Ct vs log (template copy number). Standard primers amplified the target as expected by doubling every cycle while SDCR amplified the target more than doubling per cycle. The calculated PCR and SDCR efficiencies are listed in the table below.

| Forward | Reverse | Standard Curve | Exponential | PCR or SDCR |
|---|---|---|---|---|
| Primer | Primer | Slope | Base | Efficiency |
| Standard | Ribo | −1.8763 | 3.41 | 241% |
| Ribo | Standard | −2.1419 | 2.92 | 192% |
| Ribo | Ribo | −2.4496 | 2.56 | 156% |
| Standard | Standard | −3.203 | 2.05 | 105% |

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

The steps shown in the figures, explained in the specification and recited in the claims are for illustration purposes only. It is understood that these steps do not necessarily need to be performed sequentially according to the illustrated order unless specifically required or dictated by the context.

Other embodiments and uses of the invention will be apparent to those skilled in the art from consideration of the specification and practice of the invention disclosed herein. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the claims.

1. A nucleic acid primer set comprising:
a first primer having a 3′ end and a 5′ end for amplifying a target nucleic acid in a sample, wherein the target nucleic acid includes a first template strand, and wherein the first primer comprises a first segment of DNA oligonucleotide and a second segment of DNA oligonucleotide, the first segment being at 5′ side of the second segment, the first segment and the second segment being linked by at least one first ribonucleotide, at least a portion of the second segment is capable of specifically hybridizing to the first template strand.

2. The primer set of claim 1, wherein the at least one first ribonucleotide of the first primer includes two or more ribonucleotides connected consecutively or inconsecutively.

3. The primer set of claim 1, wherein the at least one first ribonucleotide of the first primer consists of a single ribonucleotide.

4. The primer set of claim 1, wherein both the first segment and the second segment of the first primer specifically hybridize to the first template strand at a given condition.

5. The primer set of claim 1, wherein the target nucleic acid further includes a second template strand complementary to the first template strand, the primer set further comprising: a second primer capable of specifically hybridizing to the second template strand.

6. The primer set of claim 5, wherein the second primer has a 3′ end and a 5′ end and comprises a third segment of DNA oligonucleotide and a fourth segment of DNA oligonucleotide, the third segment being at 5′ side of the fourth segment, the third segment and the fourth segment being linked by at least one second ribonucleotide, at least the fourth segment is capable of specifically hybridizing to the second template strand.

7. A method for amplifying a target nucleic acid in a sample, wherein the target nucleic acid includes a first template strand, the method comprising:
(a) contacting the sample with a first primer having a 3′ end and a 5′ end and comprising a first oligonucleotide segment and a second oligonucleotide segment, the first oligonucleotide segment being at 5′ side of and linked with the second oligonucleotide segment by at least one first ribonucleotide, under conditions where at least a portion of the second segment anneals to the first template strand;
(b) producing a first amplicon complementary to the first template strand by extending the first primer using the first template strand as the template; and
(c) cleaving the first primer at the position immediately 5′ to the at least one first ribonucleotide.

8. The method of claim 7, wherein the cleaving comprises using RNase H2.

9. The method of claim 7, wherein the first segment anneals to the first template strand under the conditions, the method further comprising:
producing a second amplicon complementary to the first template strand by extending the first segment of the first primer using the first template strand as a template while replacing the first amplicon from the first template strand.

10. The method of claim 7, wherein the first segment of the first primer does not anneal to the first template strand under the conditions, the method further comprising:
prior to cleaving at (c):
separating the first amplicon from the first template strand;
using a second primer to produce a nucleic acid strand complementary to the first amplicon, the new nucleic acid strand including a segment complementary to the first segment of the first primer;
and after cleaving at (c):
producing a third amplicon complementary to the new nucleic acid strand by extending the first segment of the first primer using the new nucleic acid strand as a template, while replacing the first amplicon from the new nucleic acid strand.

11. The method of claim 10, wherein at least a portion to 3′ side of the at least one first ribonucleotide and distal to the 3′ end of the first primer does not specifically hybridize to the first template strand.

12. The method of claim 7, wherein the target nucleic acid further includes a second template strand complementary to the first template strand, the method further comprising:
(d) contacting the sample with a second primer comprising a third segment of DNA oligonucleotide and a fourth segment of DNA oligonucleotide, the third segment being at the 5′ side of and linked with the fourth segment by at least one second ribonucleotide, where at least a portion of the fourth segment anneals to the second template strand under the conditions; and
(e) producing a second amplicon complementary to the second template strand by extending the second primer using the second template strand as the template; and
(f) cleaving the second primer at the position immediately 5′ to the at least one second ribonucleotide.

13. The method of claim 7, wherein the first segment and second segment both anneal to the first template strand under the conditions, the target nucleic acid further includes a second template strand complementary to the first template strand, the method further comprising:
(d) contacting the sample with a second primer comprising a third segment of DNA oligonucleotide and a fourth segment of DNA oligonucleotide, the third segment being at the 5′ side of and linked with the fourth segment by at least one second ribonucleotide, where both the third segment and fourth segment anneal to the second template strand under the conditions;
(e) producing a second amplicon complementary to the second template strand by extending the second primer using the second template strand as the template;
(f) cleaving the second primer at the position immediately 5′ to the at least one second ribonucleotide; and
(g) producing a third amplicon complementary to the first template strand by extending the first segment of the first primer using the first template strand as a template while replacing the first amplicon from the first template strand, and producing a fourth amplicon complementary to the second template strand by extending the second primer using the second template strand as the template while replacing the third amplicon from the second template strand.

14. The method of claim 13, further comprising:
(h) producing a fifth amplicon complementary to the first amplicon using another second primer; and producing a sixth amplicon complementary to the second amplicon using another first primer.

15. The method of claim 14, further comprising:
increasing temperature to separate double-stranded amplicons produced; and
decreasing temperature to anneal further first primers and second primers to the separated amplicons.

16. The method of claim 7, wherein the first primer is anchored on a solid surface or in a matrix.

17. The method of claim 7, wherein the first template strand is an RNA.

18. The method of claim 7, further comprising:
(d) using the first segment cleaved from the first primer as a primer to initiate nucleotide polymerization using a second target nucleic acid.

19. A method of detecting a nucleotide variation in a target nucleic acid in a sample, the sample containing a first double-stranded DNA and a second double stranded DNA, the second double stranded DNA differing from the first double stranded DNA at at least one variance position, the method comprising:
(a) contacting the sample with a first primer having a 3′ end and a 5′ end and comprising a first oligonucleotide segment and a second oligonucleotide segment, the first oligonucleotide segment being at 5′ side of and linked with the second oligonucleotide segment by at least one first ribonucleotide, under conditions such that the first primer overall anneals to a first template strand of the first double-stranded DNA as well as the first template strand of the second double-stranded DNA, but the second segment of the first primer by itself does not anneal to the first template strand of the first double-stranded DNA or to the first template strand of the second double-stranded DNA, wherein the ribonucleotide of the first primer is aligned with or in the vicinity of the variance position such that the first primer is perfectly matched with the first template strand of the first double-stranded DNA but not perfectly matched with the first template strand of the second double-stranded DNA;
(b) producing a first amplicon by extending the first primer hybridized on the first template of the first double-stranded DNA using the first template strand of the first double-stranded DNA as the template, and producing a second amplicon by extending the first primer hybridized on the first template of the second double-stranded DNA using the first template strand of the second double-stranded DNA as the template;
(c) cleaving the first primer hybridized with the first template strand of the first double-stranded DNA at the position immediately 5′ to the at least one first ribonucleotide using an RNase to produce 5′ side segment cleaved from the first primer hybridized with the first template strand of the first double-stranded DNA, while not cleaving the first primer hybridized with the first template strand of the second double-stranded DNA; and
(d) conducting PCR to further amplify the first and second double-stranded DNA in the presence of the RNase to thereby differentiate the first and second double-stranded DNA by a quantity of respective amplicon products.

20. A method for amplifying a target nucleic acid in a sample, wherein the target nucleic acid includes a first template strand, the method comprising:
(a) contacting the sample with a first primer having a 3′ end and a 5′ end and comprising a first oligonucleotide segment and a second oligonucleotide segment, the first oligonucleotide segment being at 5′ side of and linked with the second oligonucleotide segment by at least one first ribonucleotide, under conditions where at least a portion of the second segment anneals to the first template strand;
(b) producing a first extension product complementary to the first template strand by extending the first primer using the first template strand as the template; and
(c) conducting PCR to produce duplicate copies of the first extension product, each of the amplicons containing a first primer;
(d) cleaving the first primers in the amplicons of the first extension product at the position immediately 5′ to the at least one first ribonucleotide to produce cleaved portions of the duplicate copies; and
(e) annealing or ligating the cleaved portions of the duplicate copies with other nucleic acid strands.

21. A molecular probe for detecting amplification of a nucleic acid, comprising:
a quencher portion, a fluorophore portion, and a linker portion linking the quencher portion and the fluorophore portion, the linker portion comprising a plurality of deoxyribonucleotides and a ribonucleotide, wherein no fluorescence is given by the fluorophore when then linker is intact.

22. A method of detecting an amplicon product in an amplification reaction system, comprising:
allowing a molecular probe of claim 21 to specifically hybridize to an amplicon product in an amplification reaction system;
cleaving the ribonucleotide in the molecular probe; and
detecting fluorescence given off by the fluorophore portion of the molecular probe.
